(19) United States
(12) Patent Application Publication
(10) Pub. No.: US 2025/0304635 A1
(43) Pub. Date: Oct. 2, 2025

(54) BIRINAPANT POLYMORPH H

(71) Applicant: Medivir AB, Huddinge (SE)

(72) Inventors: Anna Trillium AZULAY CHASNOFF, Albany, NY (US); Zachary Ormond POWERS, Albany, NY (US)

(21) Appl. No.: 18/720,352

(22) PCT Filed: Apr. 6, 2023
(86) PCT No.: PCT/EP2023/059175

§ 371 Date: Jun. 14, 2024

Related U.S. Application Data
(60) Provisional application No. 63/329,311, filed on Apr. 8, 2022.

(51) Int. Cl. C07K 14/47

(52) U.S. Cl. CPC C07K 14/4705

(57) ABSTRACT

Provided herein is a specific crystalline form of the anti-cancer drug birinapant, designated Form H. Compositions comprising Form H are disclosed, as well as methods of use thereof. Methods of preparation of the specific crystalline form are also provided. Form H is suitable for manufacturing drug product under GMP guidelines. Birinapant is a peptidomimetic of second mitochondrial-derived activator of caspases (SMAC), and inhibits proteins of the IAP (Inhibitor of Apoptosis Protein) family.

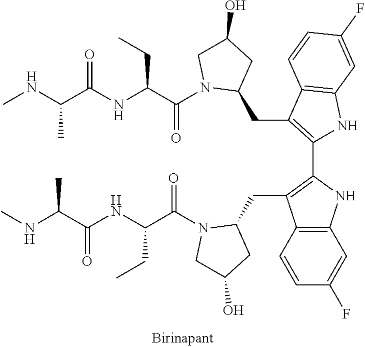 [SMILES: CC[C@H](NC(=O)[C@H](C)NC)C(=O)N1C[C@@H](O)C[C@H]1Cc1c(-c2[nH]c3cc(F)ccc3c2C[C@@H]2C[C@H](O)CN2C(=O)[C@H](CC)NC(=O)[C@H](C)NC)[nH]c2cc(F)ccc12]

### CROSS-REFERENCE TO RELATED APPLICATIONS

This application claims priority benefit of U.S. Provisional Patent Application No. 63/329,311, filed Apr. 8, 2022. The disclosure of that application is hereby incorporated herein by reference in its entirety

### FIELD

Provided herein are crystalline forms of the anti-proliferative drug birinapant, compositions thereof, and methods of use thereof.

### BACKGROUND

Cancer remains a leading cause of death with approximately 20 million new cases and 10 million deaths world-wide in 2020, according to data from the International Agency for Research on Cancer of the World Health Organization. There is an ongoing need for compounds to treat cancer and other cell proliferative disorders.

Inhibitors of Apoptosis Proteins (IAPs) are naturally occurring intra-cellular proteins that suppress caspase-dependent apoptosis. SMAC, also known as DIABLO, is another intracellular protein that functions to antagonize, i.e., inhibit the activity of IAPs. In normal healthy cells, SMAC and IAPs function together to maintain the viability of healthy cells. However, in certain disease states, e.g., cancers and other proliferative disorders, IAPs are not adequately antagonized and therefore prevent apoptosis and cause or exacerbate abnormal proliferation and survival.

SMAC mimetics, also known as IAP antagonists, are synthetic small molecules that mimic the structure and IAP antagonist activity of the four N-terminal amino acids of SMAC. When administered to animals suffering proliferative disorders, the SMAC mimetics antagonize IAPs, causing an increase in apoptosis among abnormally proliferating cells.

Birinapant ((2S,2'S)—N,N′-((2S,2'S)-((3S,3'S,5R,5′R)-((6,6′-difluoro-1H,1′H-[2,2′-biindole]-3,3′-diyl)bis(methylene))bis(3-hydroxypyrrolidine-5,1-diyl))bis(1-oxobutane-1,2-diyl))bis(2-(methylamino)propanamide), chemical structure shown below) is a SMAC mimetic which may be useful in the treatment of cell proliferative disorders such as cancer. This compound is disclosed in U.S. Pat. No. 8,283,372, the entire disclosure of which is hereby incorporated by reference herein. Other documents describing birinapant include Deng et al., Org. Process Res. Dev. 2016, 20, 242-252, and Deng et al., ACS Med. Chem. Lett. 2016, 7, 318-323, the entire disclosures of which are hereby incorporated by reference herein.

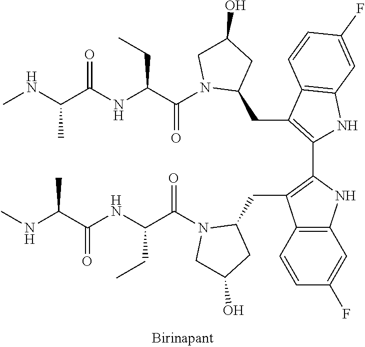 [SMILES: CC[C@H](NC(=O)[C@H](C)NC)C(=O)N1C[C@@H](O)C[C@H]1Cc1c(-c2[nH]c3cc(F)ccc3c2C[C@@H]2C[C@H](O)CN2C(=O)[C@H](CC)NC(=O)[C@H](C)NC)[nH]c2cc(F)ccc12]

Because drug compounds having, for example, improved stability, solubility, shelf life and in vivo pharmacology are constantly sought, there is an ongoing need for new forms of birinapant. The crystalline forms, preparative methods, and formulations described herein help to meet this need.

### SUMMARY

Provided herein is a crystalline Form H of birinapant.

Also provided is a method of preparing birinapant Form H comprising converting a first form of birinapant (Form D, an ethyl acetate solvate of birinapant) to a second form of birinapant via a conversion process, wherein the second form of birinapant is Form H. In some variations, the method further comprises preparing the first form (Form D) of birinapant via a formation process.

Also provided are compositions comprising birinapant Form H.

Also provided are methods of using compositions comprising birinapant Form H for use in treating cell proliferative disorders (e.g., cancer).

### BRIEF DESCRIPTION OF THE DRAWINGS

FIG. 1A shows experimental X-ray powder diffraction (XRPD) pattern of crystalline form H of birinapant.

FIG. 1B shows another experimental X-ray powder diffraction (XRPD) pattern of crystalline form H of birinapant.

FIG. 2 shows an 1H NMR spectrum of birinapant form H in DMSO.

FIG. 3A shows a differential scanning calorimetry (DSC) graph of birinapant form H

FIG. 3B shows a variable temperature X-ray powder diffraction (VT-XRPD) graph for birinapant form H.

FIG. 4 shows a thermogravametric analysis (TGA) graph of birinapant form H.

FIG. 5 shows a dynamic vapor sorption (DVS) graph of birinapant form H.

FIG. 6 shows an XRPD pattern comparison of birinapant Form stressed at 60° C. View (a) of FIG. 6 shows the pattern from 0° angle 20 to 40° angle 20, while view (b) of FIG. 6 shows the pattern from 0° angle 20 to 20° angle 20 in more detail.

FIG. 7 shows an XRPD pattern comparison of birinapant Form stressed at 30° C./62% relative humidity.

FIG. 8 shows an XRPD pattern comparison of birinapant Form H stressed at 40° C./75% relative humidity.

FIG. 9 shows an XRPD pattern comparison of birinapant Form H stressed at 60° C., 30° C./62% relative humidity, or 40° C./75% relative humidity.

### DETAILED DESCRIPTION

### Definitions

As used herein, unless clearly indicated otherwise, use of the terms “a”, “an” and the like refers to one or more.

As used herein, reference to “about” a value or parameter herein includes (and describes) embodiments that are directed to that value or parameter per se. For example, description referring to “about X” includes description of “X”. As used herein, and unless otherwise specified, the terms “about” and “approximately,” when used in connection with doses, amounts, or weight percent of ingredients of a composition or a dosage form, mean a dose, amount, or weight percent that is recognized by those of ordinary skill in the art to provide a pharmacological effect equivalent to that obtained from the specified dose, amount, or weight percent. Specifically, the terms “about” and “approximately,” when used in this context, contemplate a dose, amount, or weight percent within 20%, within 15%, within 10%, within 5%, within 4%, within 3%, within 2%, within 1%, or within 0.5% of the specified dose, amount, or weight percent.

As used herein, the term “crystalline form” refers to a crystalline solid form of a chemical compound, including, but not limited to, a single-component or multiple-component crystal form, e.g., a polymorph of a compound; or a solvate, a hydrate, a clathrate, a cocrystal, a salt of a compound, or a polymorph thereof. The term “crystal forms” and related terms herein refers to the various crystalline modifications of a given substance, including, but not limited to, polymorphs, solvates, hydrates, co-crystals and other molecular complexes, as well as salts, solvates of salts, hydrates of salts, other molecular complexes of salts, and polymorphs thereof. Crystal forms of a substance can be obtained by a number of methods, as known in the art. Such methods include, but are not limited to, melt recrystallization, melt cooling, solvent recrystallization, slurrying, recrystallization in confined spaces such as, e.g., in nanopores or capillaries, recrystallization on surfaces or templates such as, e.g., on polymers, recrystallization in the presence of additives, such as, e.g., anti-solvents, co-crystal counter-molecules, desolvation, dehydration, rapid evaporation, rapid cooling, slow cooling, vapor diffusion, sublimation, grinding and solvent-drop grinding.

Unless clearly indicated otherwise, “an individual” as used herein intends a mammal, including but not limited to a primate, human, bovine, horse, feline, canine, or rodent. In one variation, the individual is a human.

As used herein, “treatment” or “treating” is an approach for obtaining beneficial or desired results, including clinical results. Beneficial or desired results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, delaying the occurrence or recurrence of the disease, delay or slowing the progression of the disease, ameliorating the disease state, providing a remission (whether partial or total) of the disease, decreasing the dose of one or more other medications required to treat the disease, enhancing effect of another medication, delaying the progression of the disease, increasing the quality of life, and/or prolonging survival. The methods of the invention contemplate any one or more of these aspects of treatment.

As used herein, the term “effective amount” intends such amount of a compound of the invention which should be effective in a given therapeutic form. As is understood in the art, an effective amount may be in one or more doses, i.e., a single dose or multiple doses may be required to achieve the desired treatment endpoint. An effective amount may be considered in the context of administering one or more therapeutic agents (e.g., a compound, or pharmaceutically acceptable salt thereof), and a single agent may be considered to be given in an effective amount if, in conjunction with one or more other agents, a desirable or beneficial result may be or is achieved. Suitable doses of any of the co-administered compounds may optionally be lowered due to the combined action (e.g., additive or synergistic effects) of the compounds.

As used herein, a “therapeutically effective amount” refers to an amount of a compound or salt thereof sufficient to produce a desired therapeutic outcome.

As used herein, “unit dosage form” refers to physically discrete units, suitable as unit dosages, each unit containing a predetermined quantity of active ingredient calculated to produce the desired therapeutic effect in association with the required pharmaceutical carrier. Unit dosage forms may contain a single or a combination therapy.

As used herein, the term “controlled release” refers to a drug-containing formulation or fraction thereof in which release of the drug is not immediate, i.e., with a “controlled release” formulation, administration does not result in immediate release of the drug into an absorption pool. The term encompasses depot formulations designed to gradually release the drug compound over an extended period of time. Controlled release formulations can include a wide variety of drug delivery systems, generally involving mixing the drug compound with carriers, polymers or other compounds having the desired release characteristics (e.g., pH-dependent or non-pH-dependent solubility, different degrees of water solubility, and the like) and formulating the mixture according to the desired route of delivery (e.g., coated capsules, implantable reservoirs, injectable solutions containing biodegradable capsules, and the like).

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethyl cellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

Unless otherwise stated, “substantially pure” intends a composition that contains no more than about 10% impurity, such as a composition comprising less than about 9%, about 7%, about 5%, about 3%, about 1%, or about 0.5% impurity.

It is understood that aspects and embodiments described herein as “comprising” include “consisting of” and “consisting essentially of” embodiments.

As used herein, the term “substantially as shown in” when referring, for example, to an XRPD pattern, a DSC graph, a TGA graph, or a DVS graph, includes a pattern or graph that is not necessarily identical to those depicted herein, but that falls within the limits of experimental error or deviations when considered by one of ordinary skill in the art.

### Crystalline Forms

In one aspect, provided herein is a crystalline form of birinapant, a compound having the chemical name (2S,2'S)—N,N′-((2S,2'S)-((3S,3'S,5R,5′R)-((6,6′-difluoro-1H,1′H-[2,2′-biindole]-3,3′-diyl)bis(methylene))bis(3-hydroxypyrrolidine-5,1-diyl))bis(1-oxobutane-1,2-diyl))bis(2-(methylamino)propanamide), or a compound having the chemical structure shown below:

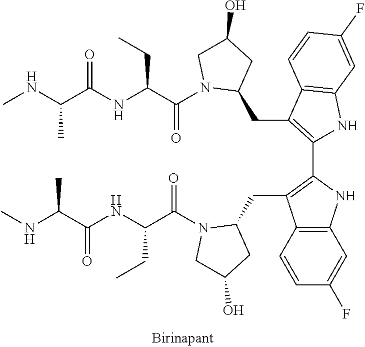 [SMILES: CC[C@H](NC(=O)[C@H](C)NC)C(=O)N1C[C@@H](O)C[C@H]1Cc1c(-c2[nH]c3cc(F)ccc3c2C[C@@H]2C[C@H](O)CN2C(=O)[C@H](CC)NC(=O)[C@H](C)NC)[nH]c2cc(F)ccc12]

As used herein, a reference to the “parent compound” means the salt-free form of birinapant. Likewise, reference to “birinapant” alone or its structural formula alone will, unless otherwise noted or made clear in the context in which the reference is used, be a reference to the parent compound.

In another aspect, provided herein is a crystalline form of birinapant referred to herein as “crystalline H Form of birinapant”, “crystalline Form H”, “birinapant Form H”, “H Form of birinapant”, “birinapant H Form”, or simply “Form H”.

The crystalline H Form of birinapant disclosed herein may provide the advantages of bioavailability and stability and may be suitable for use as an active agent in a pharmaceutical composition. Variations in the crystal structure of a pharmaceutical drug substance may affect the dissolution rate (which may affect bioavailability, etc.), manufacturability (e.g., ease of handling, ease of purification, ability to consistently prepare doses of known strength, etc.) and stability (e.g., thermal stability, shelf life (including resistance to degradation), etc.) of a pharmaceutical drug product. Such variations may affect the methods of preparation or formulation of pharmaceutical compositions in different dosage or delivery forms, such as solid oral dosage forms including tablets and capsules. Compared to other crystalline forms, non-crystalline forms, or amorphous forms, the H Form of birinapant may provide desired or suitable hygroscopicity, particle size control, dissolution rate, solubility, purity, physical and chemical stability, manufacturability, yield, reproducibility, and/or process control. Thus, the crystalline H Form of birinapant disclosed herein may provide advantages of improving the manufacturing process of an active agent or the stability or storability of a drug product form of the active agent, or having suitable bioavailability and/or stability as an active agent.

In some embodiments, crystalline Form H of birinapant is stable at ambient temperature and humidity. In some embodiments, crystalline Form H of birinapant exhibits a level of stability at ambient temperature and humidity that is sufficient for use in the manufacture of pharmaceutical formulations. In some embodiments, Form H is stable over a specified period of time, under a specified set of environmental conditions. In some embodiments, the specified period of time is least about 1 day, at least about 5 days, at least about 10 days, at least about 17 days, at least about 18 days, at least about 1 month, at least about 33 days, at least about 2 months, at least about 3 months, at least about 105 days, at least about 4 months, at least about 5 months, at least about 6 months, at least about 9 months, at least about 12 months, or at least about 18 months. In some embodiments, the specified environmental conditions include temperature. In some such embodiments, the temperature is about 10° C., about 20° C., about 30° C., about 40° C., about 50° C., about 60° C., about 70° C., or about 80° C. In some embodiments, the specified environmental condition includes relative humidity. In some such embodiments, the relative humidity is about 0%, about 10%, about 20%, about 30%, about 40%, about 50%, about 53%, about 55%, about 60%, about 62%, about 65%, about 70%, about 75%, about 80%, about 85%, about 90%, or about 100%.

In some embodiments, crystalline Form H of birinapant is stable at about 40° C. and about 75% relative humidity for at least about 6 months. In some embodiments, crystalline Form H of birinapant is stable at about 20° C. and about 53% relative humidity for at least about 18 months.

In some embodiments, crystalline Form H of birinapant is stable at about 60° C. for at least about 17 days, at least about 33 days, or at least about 105 days. In some embodiments, crystalline Form H of birinapant is stable at about 80° C. for at least about 17 days or at least about 33 days. In some embodiments, Form H of birinapant is stable at about 20° C. and about 53% relative humidity for at least about 18 days. In some embodiments, Form H of birinapant is stable at about 30° C. and about 62% relative humidity for at least about 17 days, at least about 33 days, or at least about 105 days. In some embodiments, Form H of birinapant is stable at about 40° C. and about 75% relative humidity for at least about 17 days, at least about 33 days, or at least about 105 days. In some embodiments, Form H of birinapant is stable at about 20° C. and about 98% relative humidity for at least about 18 days.

In some embodiments, crystalline form H of birinapant is stable at about 60° C. for at least about 182 days. In some embodiments, crystalline form H of birinapant is stable at about 60° C. for about 182 days. In some embodiments, crystalline form H of birinapant is stable at about 30° C. and about 62% relative humidity for at least about 182 days. In some embodiments, crystalline form H of birinapant is stable at about 30° C. and about 62% relative humidity for about 182 days. In some embodiments, crystalline form H of birinapant is stable at about 40° C. and about 75% relative humidity for at least about 182 days. In some embodiments, crystalline form H of birinapant is stable at about 40° C. and about 75% relative humidity for about 182 days.

In some embodiments, crystalline form H of birinapant is stable at about 60° C. for at least about 6 months. In some embodiments, crystalline form H of birinapant is stable at about 60° C. for about 6 months. In some embodiments, crystalline form H of birinapant is stable at about 30° C. and about 62% relative humidity for at least about 6 months. In some embodiments, crystalline form H of birinapant is stable at about 30° C. and about 62% relative humidity for about 6 months. In some embodiments, crystalline form H of birinapant is stable at about 40° C. and about 75% relative humidity for at least about 6 months. In some embodiments, crystalline form H of birinapant is stable at about 40° C. and about 75% relative humidity for about 6 months.

Techniques for characterizing polymorphs include x-ray powder diffraction (XRPD), single crystal x-ray diffraction (XRD), differential scanning calorimetry (DSC), vibrational spectroscopy (e.g., IR and Raman spectroscopy), solid state nuclear magnetic resonance (ssNMR), hot stage optical microscopy, scanning electron microscopy (SEM), electron crystallography and quantitative analysis, particle size analysis (PSA), surface area analysis, solubility studies, and dissolution studies.

### Crystalline Form H of Birinapant

In some embodiments, provided herein is a crystalline form of birinapant (Form H).

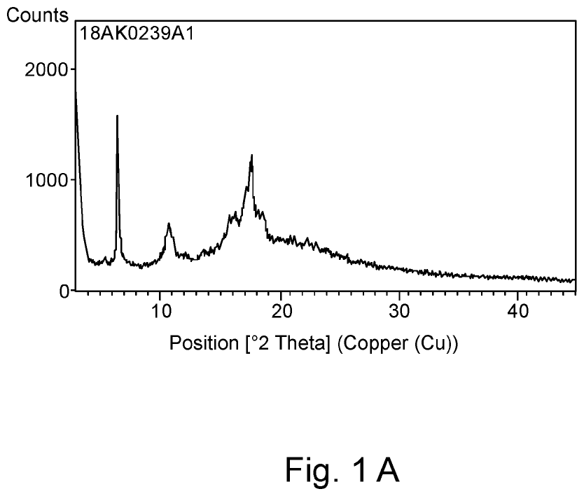

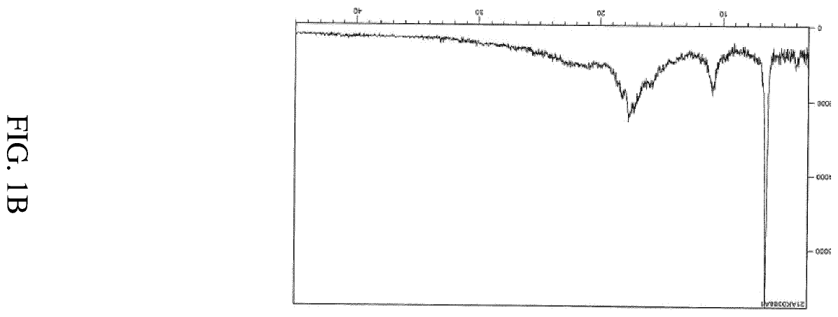

In some embodiments, Form H has an XRPD pattern substantially as shown in FIG. 1A. Positions of peaks and relative peak intensities that may be observed for the crystalline form using XRPD are shown in Table 1. The peak positions in Table 1 are given in units of degrees two theta (° 2Th.). The heights of the peaks are given as the number of counts (cts.). Also provided in Table 1 are the full width of the peak at half the maximum peak height (FWHM), the d-spacing of each peak in angstroms (Å), the relative intensity as a percent of the most intense peak, and the tip width of each peak in degrees two theta (°2Th.).

TABLE 1

| | | | | Relative | |
|---|---|---|---|---|---|
| Position | | FWHM | d-spacing | Intensity | Tip width |
| [°2Th.] | Height [cts.] | [°2Th.] | [Å] | [%] | [°2Th.] |
| 3.1670 | 328.88 | 0.0394 | 27.89815 | 4.55 | 0.0472 |
| 4.0309 | 785.64 | 0.0787 | 21.92100 | 10.88 | 0.0945 |
| 4.3478 | 462.81 | 0.0590 | 20.32403 | 6.41 | 0.0708 |
| 4.4904 | 398.59 | 0.0394 | 19.67880 | 5.52 | 0.0472 |
| 4.6950 | 498.39 | 0.0394 | 18.82158 | 6.90 | 0.0472 |
| 4.9640 | 457.26 | 0.1181 | 17.80232 | 6.33 | 0.1417 |
| 5.0908 | 481.23 | 0.0984 | 17.35930 | 6.66 | 0.1181 |
| 5.4009 | 427.53 | 0.1574 | 16.36302 | 5.92 | 0.1889 |
| 5.8062 | 538.13 | 0.0590 | 15.22176 | 7.45 | 0.0708 |
| 6.0894 | 726.45 | 0.0394 | 14.51439 | 10.06 | 0.0472 |
| 6.4732 | 7223.58 | 0.1378 | 13.65479 | 100.00 | 0.1653 |
| 7.1491 | 559.05 | 0.0590 | 12.36530 | 7.74 | 0.0708 |
| 8.0707 | 422.88 | 0.0787 | 10.95524 | 5.85 | 0.0945 |
| 8.7735 | 279.53 | 0.3149 | 10.07910 | 3.87 | 0.3779 |
| 9.0057 | 220.94 | 0.0787 | 9.81982 | 3.06 | 0.0945 |
| 9.1483 | 366.39 | 0.0984 | 9.66704 | 5.07 | 0.1181 |
| 10.0359 | 580.29 | 0.0394 | 8.81398 | 8.03 | 0.0472 |
| 10.6682 | 1207.86 | 0.0590 | 8.29296 | 16.72 | 0.0708 |
| 10.8201 | 1364.14 | 0.1574 | 8.17681 | 18.88 | 0.1889 |
| 12.6589 | 426.50 | 0.0590 | 6.99293 | 5.90 | 0.0708 |
| 13.2403 | 453.64 | 0.1574 | 6.68715 | 6.28 | 0.1889 |
| 13.9756 | 562.68 | 0.3936 | 6.33691 | 7.79 | 0.4723 |
| 15.0402 | 831.76 | 0.1181 | 5.89069 | 11.51 | 0.1417 |
| 15.9382 | 1193.96 | 0.1181 | 5.56075 | 16.53 | 0.1417 |
| 16.1873 | 1315.52 | 0.0590 | 5.47575 | 18.21 | 0.0708 |
| 17.6709 | 2179.19 | 0.0787 | 5.01921 | 30.17 | 0.0945 |
| 18.1986 | 1465.93 | 0.1968 | 4.87483 | 20.29 | 0.2362 |
| 18.6680 | 1234.45 | 0.0590 | 4.75331 | 17.09 | 0.0708 |
| 21.0399 | 717.70 | 0.6298 | 4.22251 | 9.94 | 0.7557 |
| 22.8822 | 607.25 | 0.7872 | 3.88654 | 8.41 | 0.9446 |
| 23.6266 | 527.86 | 0.2362 | 3.76575 | 7.31 | 0.2834 |
| 24.7027 | 412.01 | 0.1181 | 3.60409 | 5.70 | 0.1417 |
| 25.5650 | 351.52 | 0.2362 | 3.48445 | 4.87 | 0.2834 |
| 26.1603 | 335.41 | 0.0590 | 3.40650 | 4.64 | 0.0708 |
| 28.3855 | 167.02 | 0.4723 | 3.14431 | 2.31 | 0.5668 |
| 29.5919 | 125.38 | 0.4723 | 3.01881 | 1.74 | 0.5668 |
| 30.2191 | 160.82 | 0.0590 | 2.95758 | 2.23 | 0.0708 |
| 31.5809 | 75.59 | 0.2362 | 2.83308 | 1.05 | 0.2834 |
| 40.8369 | 26.07 | 1.1520 | 2.20796 | 0.36 | 1.3824 |

In some embodiments, Form H has an XRPD pattern comprising one or more (e.g., at least one, at least two, at least three, at least four, at least five, at least six, at least seven, at least eight, at least nine, or at least ten) of the peaks at angles 2-theta with the greatest intensity in the XRPD pattern substantially as shown in FIG. 1A, or as provided in Table 1. In some embodiments, Form H has an XRPD pattern comprising the peaks provided in Table 1. It should be understood that relative intensities and peak assignments can vary depending on a number of factors, including sample preparation, mounting, the instrument and analytical procedure and settings used to obtain the spectrum, temperature effects on the unit cell, and extent of solvation, e.g., hydration, of the sample. For example, relative peak intensities and peak assignments can vary within experimental error. In some embodiments, each peak assignment listed herein, including for Form H, can independently vary by ±0.6 degrees, ±0.4 degrees, ±0.2 degrees, or ±0.1 degrees 2-theta. In some embodiments, each peak assignment listed herein, including for Form H, can independently vary by ±0.2 degrees 2-theta.

In some embodiments, Form H has an XRPD pattern comprising a peak assigned at an angle 2-theta in degrees of about 6.47. In some embodiments, Form H has an XRPD pattern comprising a peak assigned at an angle 2-theta in degrees of about 6.47±0.2.

In some embodiments, Form H has an XRPD pattern comprising peaks assigned at angles 2-theta in degrees of about 6.47, about 17.67, and about 18.20. In some embodiments, Form H has an XRPD pattern comprising peaks each assigned at an angle 2-theta in degrees of about 6.47±0.2, about 17.67±0.2, and about 18.20±0.2.

In some embodiments, Form H has an XRPD pattern comprising peaks each assigned at an angle 2-theta in degrees of about 6.47 (e.g., about 6.47±0.2), about 10.67 (e.g., about 10.67±0.2), about 10.82 (e.g., about 10.82±0.2), about 15.94 (e.g., about 15.94±0.2), about 16.19 (e.g., about 16.19±0.2), about 17.67 (e.g., about 17.67±0.2), about 18.20 (e.g., about 18.20±0.2), and about 18.67 (e.g., about 18.67±0.2). In some embodiments, Form H has an XRPD pattern comprising one or more (e.g., at least one, at least two, at least three, at least four, at least five, at least six, or at least seven) peaks each assigned at angles 2-theta in degrees of about: 6.47 (e.g., about 6.47±0.2), about 10.67 (e.g., about 10.67±0.2), about 10.82 (e.g., about 10.82±0.2), about 15.94 (e.g., about 15.94±0.2), about 16.19 (e.g., about 16.19±0.2), about 17.67 (e.g., about 17.67±0.2), about 18.20 (e.g., about 18.20±0.2), and about 18.67 (e.g., about 18.67±0.2). In some embodiments, Form H has an XRPD pattern comprising peaks each assigned at an angle 2-theta in degrees of about 6.47, about 10.67, about 10.82, about 15.94, about 16.19, about 17.67, about 18.20, and about 18.67. In some embodiments, Form H has an XRPD pattern comprising peaks each assigned at an angle 2-theta in degrees of about 6.47±0.2, about 10.67±0.2, about 10.82±0.2, about 15.94±0.2, about 16.19±0.2, about 17.67±0.2, about 18.20±0.2, and about 18.67±0.2.

In some embodiments, Form H has an XRPD pattern comprising one or more (e.g., at least one, at least two, at least three, at least four, at least five, at least six, at least seven, at least eight, at least nine, at least ten, or eleven) peaks each assigned at angles 2-theta in degrees of about: 4.03 (e.g., about 4.03±0.2), about 6.09 (e.g., about 6.09±0.2), about 6.47 (e.g., about 6.47±0.2), about 10.67 (e.g., about 10.67±0.2), about 10.82 (e.g., about 10.82±0.2), about 15.04 (e.g., about 15.04±0.2), about 15.94 (e.g., about 15.94±0.2), about 16.19 (e.g., about 16.19±0.2), about 17.67 (e.g., about 17.67±0.2), about 18.20 (e.g., about 18.20±0.2), and about 18.67 (e.g., about 18.67±0.2). In some embodiments, Form H has an XRPD pattern comprising peaks each assigned at an angle 2-theta in degrees of about 4.03 (e.g., about 4.03±0.2), about 6.09 (e.g., about 6.09±0.2), about 6.47 (e.g., about 6.47±0.2), about 10.67 (e.g., about 10.67±0.2), about 10.82 (e.g., about 10.82±0.2), about 15.04 (e.g., about 15.04±0.2), about 15.94 (e.g., about 15.94±0.2), about 16.19 (e.g., about 16.19±0.2), about 17.67 (e.g., about 17.67±0.2), about 18.20 (e.g., about 18.20±0.2), and about 18.67 (e.g., about 18.67±0.2).

In some embodiments, Form H has an XRPD pattern comprising peaks as assigned at angles 2-theta in degrees as recited in Table 1, each peak of which can independently vary in assignment at angle 2-theta in degrees as described herein. In some embodiments, Form H has an XRPD pattern comprising one or more (e.g., at least one, at least two, at least three, at least four, at least five, at least six, at least seven, at least eight, at least nine, or at least ten) peaks each assigned at angles 2-theta in degrees of about: 3.17 (e.g., about 3.17±0.2), about 4.03 (e.g., about 4.03±0.2), about 4.35 (e.g., about 4.35±0.2), about 4.49 (e.g., about 4.49±0.2), about 4.70 (e.g., about 4.70±0.2), about 4.96 (e.g., about 4.96±0.2), about 5.09 (e.g., about 5.09±0.2), about 5.40 (e.g., about 5.40±0.2), about 5.81 (e.g., about 5.81±0.2), about 6.09 (e.g., about 6.09±0.2), about 6.47 (e.g., about 6.47±0.2), about 7.15 (e.g., about 7.15±0.2), about 8.07 (e.g., about 8.07±0.2), about 8.77 (e.g., about 8.77±0.2), about 9.01 (e.g., about 9.01±0.2), about 9.15 (e.g., about 9.15±0.2), about 10.04 (e.g., about 10.04±0.2), about 10.67 (e.g., about 10.67±0.2), about 10.82 (e.g., about 10.82±0.2), about 12.66 (e.g., about 12.66±0.2), about 13.24 (e.g., about 13.24±0.2), about 13.98 (e.g., about 13.98±0.2), about 15.04 (e.g., about 15.04±0.2), about 15.94 (e.g., about 15.94±0.2), about 16.19 (e.g., about 16.19±0.2), about 17.67 (e.g., about 17.67±0.2), about 18.20 (e.g., about 18.20±0.2), about 18.67 (e.g., about 18.67±0.2), about 21.04 (e.g., about 21.04±0.2), about 22.88 (e.g., about 22.88±0.2), about 23.63 (e.g., about 23.63±0.2), about 24.70 (e.g., about 24.70±0.2), about 25.57 (e.g., about 25.57±0.2), about 26.16 (e.g., about 26.16±0.2), about 28.39 (e.g., about 28.39±0.2), about 29.59 (e.g., about 29.59±0.2), about 30.22 (e.g., about 30.22±0.2), about 31.58 (e.g., about 31.58±0.2), and about 40.84 (e.g., about 40.84±0.2). In some embodiments, Form H may have an XRPD pattern comprising peaks each assigned at an angle 2-theta in degrees of about 3.17 (e.g., about 3.17±0.2), about 4.03 (e.g., about 4.03±0.2), about 4.35 (e.g., about 4.35±0.2), about 4.49 (e.g., about 4.49±0.2), about 4.70 (e.g., about 4.70±0.2), about 4.96 (e.g., about 4.96±0.2), about 5.09 (e.g., about 5.09±0.2), about 5.40 (e.g., about 5.40±0.2), about 5.81 (e.g., about 5.81±0.2), about 6.09 (e.g., about 6.09±0.2), about 6.47 (e.g., about 6.47±0.2), about 7.15 (e.g., about 7.15±0.2), about 8.07 (e.g., about 8.07±0.2), about 8.77 (e.g., about 8.77±0.2), about 9.01 (e.g., about 9.01±0.2), about 9.15 (e.g., about 9.15±0.2), about 10.04 (e.g., about 10.04±0.2), about 10.67 (e.g., about 10.67±0.2), about 10.82 (e.g., about 10.82±0.2), about 12.66 (e.g., about 12.66±0.2), about 13.24 (e.g., about 13.24±0.2), about 13.98 (e.g., about 13.98±0.2), about 15.04 (e.g., about 15.04±0.2), about 15.94 (e.g., about 15.94±0.2), about 16.19 (e.g., about 16.19±0.2), about 17.67 (e.g., about 17.67±0.2), about 18.20 (e.g., about 18.20±0.2), about 18.67 (e.g., about 18.67±0.2), about 21.04 (e.g., about 21.04±0.2), about 22.88 (e.g., about 22.88±0.2), about 23.63 (e.g., about 23.63±0.2), about 24.70 (e.g., about 24.70±0.2), about 25.57 (e.g., about 25.57±0.2), about 26.16 (e.g., about 26.16±0.2), about 28.39 (e.g., about 28.39±0.2), about 29.59 (e.g., about 29.59±0.2), about 30.22 (e.g., about 30.22±0.2), about 31.58 (e.g., about 31.58±0.2), and about 40.84 (e.g., about 40.84±0.2).

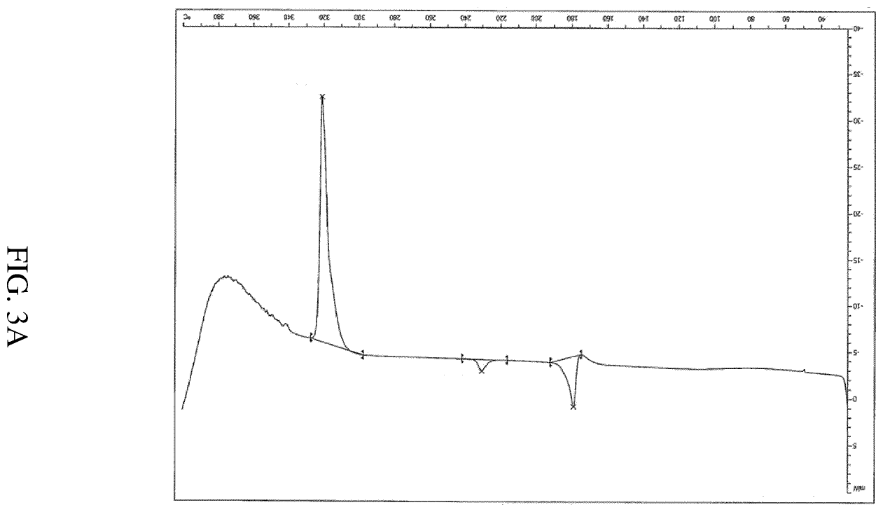

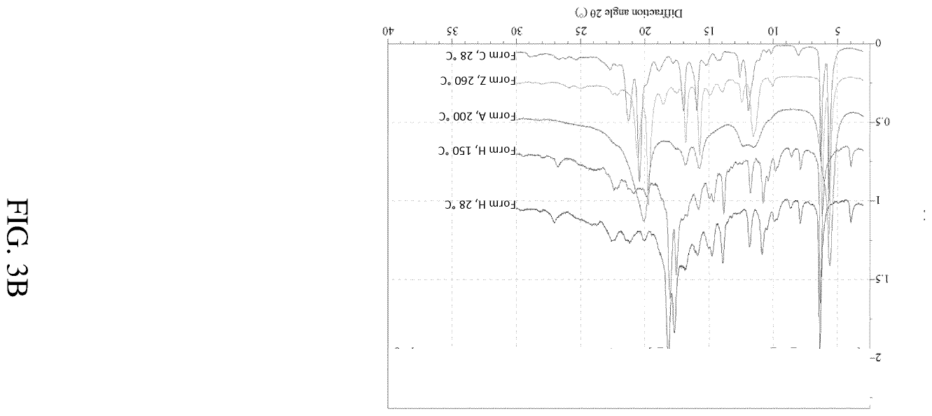

In some embodiments, Form H has a DSC graph substantially as shown in FIG. 3A. In some embodiments, Form H is characterized as having an endotherm peak at about 175° C., as determined by DSC. In some embodiments, Form H is characterized as having an exotherm peak at about 180° C., as determined by DSC. In some embodiments, Form H has a melting point between 171° C. and about 177° C.

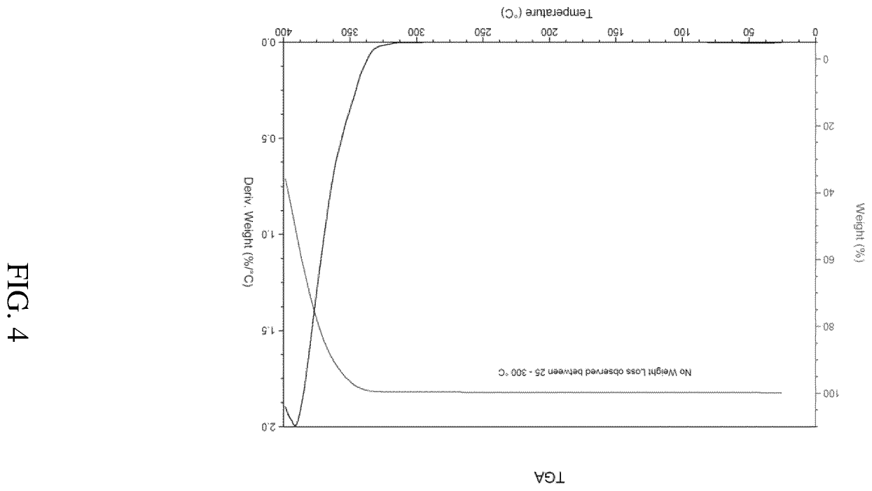

In some embodiments, Form H has a TGA graph substantially as shown in FIG. 4. In some embodiments, Form H is characterized as showing substantially no weight loss after heating from about 25° C. to about 300° C., as determined by TGA.

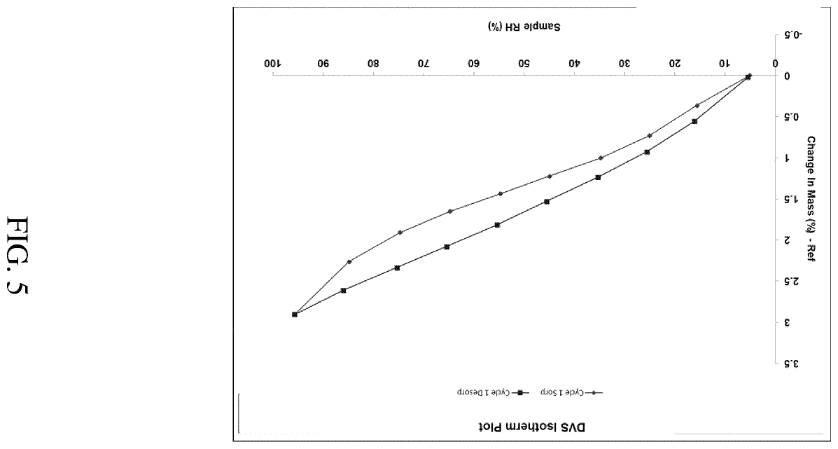

In some embodiments, Form H has a DVS graph substantially as shown in FIG. 5.

In some embodiments of Form H, at least one, at least two, at least three, at least four, at least five, at least six, or all of the following (a)-(g) apply:
(a) Form H has an XRPD pattern comprising
   (i) peaks at angles 2-theta of about 6.47 (e.g., about 6.47±0.2), about 10.67 (e.g., about 10.67±0.2), about 10.82 (e.g., about 10.82±0.2), about 15.94 (e.g., about 15.94±0.2), about 16.19 (e.g., about 16.19±0.2), about 17.67 (e.g., about 17.67±0.2), about 18.20 (e.g., about 18.20±0.2), and about 18.67 (e.g., about 18.67±0.2) degrees,
   (ii) peaks at angles 2-theta of about 4.03 (e.g., about 4.03±0.2), about 6.09 (e.g., about 6.09±0.2), about 6.47 (e.g., about 6.47±0.2), about 10.67 (e.g., about 10.67±0.2), about 10.82 (e.g., about 10.82±0.2), about 15.04 (e.g., about 15.04±0.2), about 15.94 (e.g., about 15.94±0.2), about 16.19 (e.g., about 16.19±0.2), about 17.67 (e.g., about 17.67±0.2), about 18.20 (e.g., about 18.20±0.2), and about 18.67 (e.g., about 18.67±0.2) degrees, or
   (iii) peaks at angles 2-theta of about 3.17 (e.g., about 3.17±0.2), about 4.03 (e.g., about 4.03±0.2), about 4.35 (e.g., about 4.35±0.2), about 4.49 (e.g., about 4.49±0.2), about 4.70 (e.g., about 4.70±0.2), about 4.96 (e.g., about 4.96±0.2), about 5.09 (e.g., about 5.09±0.2), about 5.40 (e.g., about 5.40±0.2), about 5.81 (e.g., about 5.81±0.2), about 6.09 (e.g., about 6.09±0.2), about 6.47 (e.g., about 6.47±0.2), about 7.15 (e.g., about 7.15±0.2), about 8.07 (e.g., about 8.07±0.2), about 8.77 (e.g., about 8.77±0.2), about 9.01 (e.g., about 9.01±0.2), about 9.15 (e.g., about 9.15±0.2), about 10.04 (e.g., about 10.04±0.2), about 10.67 (e.g., about 10.67±0.2), about 10.82 (e.g., about 10.82±0.2), about 12.66 (e.g., about 12.66±0.2), about 13.24 (e.g., about 13.24±0.2), about 13.98 (e.g., about 13.98±0.2), about 15.04 (e.g., about 15.04±0.2), about 15.94 (e.g., about 15.94±0.2), about 16.19 (e.g., about 16.19±0.2), about 17.67 (e.g., about 17.67±0.2), about 18.20 (e.g., about 18.20±0.2), about 18.67 (e.g., about 18.67±0.2), about 21.04 (e.g., about 21.04±0.2), about 22.88 (e.g., about 22.88±0.2), about 23.63 (e.g., about 23.63±0.2), about 24.70 (e.g., about 24.70±0.2), about 25.57 (e.g., about 25.57±0.2), about 26.16 (e.g., about 26.16±0.2), about 28.39 (e.g., about 28.39±0.2), about 29.59 (e.g., about 29.59±0.2), about 30.22 (e.g., about 30.22±0.2), about 31.58 (e.g., about 31.58±0.2), and about 40.84 (e.g., about 40.84±0.2) degrees;
(b) Form H has an XRPD pattern substantially as shown in FIG. 1A;
(c) Form H has a DSC graph substantially as shown in FIG. 3A;
(e) Form H has a TGA graph substantially as shown in FIG. 4;
(f) Form H is characterized as showing no weight loss after heating from about 25° C. about 300° C., as determined by TGA; and
(g) Form H has a DVS graph substantially as shown in FIG. 5.

In some embodiments of a crystalline form disclosed herein, the crystalline form is substantially anhydrous. For example, in some embodiments, the crystalline form has a water content of less than about 1%, less than about 0.5%, less than about 0.4%, less than about 0.3%, less than about 0.2%, or less than about 0.1% by weight. In some embodiments, the crystalline form has a water content in % by weight of one of about 1, 1.5, 2, 2.5, 3, 3.5, 4, 4.5 5, 5.5, 6, 6.5, 7, 7.5 8, 8.5, 9, 9.5, or 10, or a range between any two of the preceding values. For example, the crystalline form can have a water content of about 1-10 wt %. The water content can be about 2-10 wt %. The water content can be about 3-10 wt %. The water content can be about 4-10 wt %. The water content can be about 5-10 wt %. The water content can be about 6-10 wt %. The water content can be about 7-10 wt %. The water content can be about 8-10 wt %. The water content can be about 9-10 wt %. The water content can be about 1-9 wt %. The water content can be about 2-9 wt %. The water content can be about 3-9 wt %. The water content can be about 4-9 wt %. The water content can be about 5-9 wt %. The water content can be about 6-9 wt %. The water content can be about 7-9 wt %. The water content can be about 8-9 wt %. The water content can be about 1-8 wt %. The water content can be about 2-8 wt %. The water content can be about 3-8 wt %. The water content can be about 4-8 wt %. The water content can be about 5-8 wt %. The water content can be about 6-8 wt %. The water content can be about 7-8 wt %. The water content can be about 1-7 wt %. The water content can be about 2-7 wt %. The water content can be about 3-7 wt %. The water content can be about 4-7 wt %. The water content can be about 5-7 wt %. The water content can be about 6-7 wt %. The water content can be about 1-6 wt %. The water content can be about 2-6 wt %. The water content can be about 3-6 wt %. The water content can be about 4-6 wt %. The water content can be about 5-6 wt %. The water content can be about 1-5 wt %. The water content can be about 2-5 wt %. The water content can be about 3-5 wt %. The water content can be about 4-5 wt %. The water content can be about 1-4 wt %. The water content can be about 2-4 wt %. The water content can be about 3-4 wt %. The crystalline form can have a water content that varies in % by weight of ±1, ±0.75, ±0.5, ±0.25, or ±0.1 around a % by weight of one of 1, 1.5, 2, 2.5, 3, 3.5, 4, 4.5 5, 5.5, 6, 6.5, 7, 7.5 8, 8.5, 9, 9.5, or 10. For example, the crystalline form can have a water content of about 1±0.75 wt %. The water content can be about 1±0.5 wt %. The water content can be about 1±0.25 wt %. The water content can be about 1±0.1 wt %. The water content can be about 1.5±1 wt %. The water content can be about 1.5±0.75 wt %. The water content can be about 1.5±0.5 wt %. The water content can be about 1.5±0.25 wt %. The water content can be about 1.5±0.1 wt %. The water content can be about 2±1 wt %. The water content can be about 2±0.75 wt %. The water content can be about 2±0.5 wt %. The water content can be about 2±0.25 wt %. The water content can be about 2±0.1 wt %. The water content can be about 2.5±1 wt %. The water content can be about 2.5±0.75 wt %. The water content can be about 2.5±0.5 wt %. The water content can be about 2.5±0.25 wt %. The water content can be about 2.5±0.1 wt %. The water content can be about 3±1 wt %. The water content can be about 3±0.75 wt %. The water content can be about 3±0.5 wt %. The water content can be about 3±0.25 wt %. The water content can be about 3±0.1 wt %. The water content can be about 3.5±1 wt %. The water content can be about 3.5±0.75 wt %. The water content can be about 3.5±0.5 wt %. The water content can be about 3.5±0.25 wt %. The water content can be about 3.5±0.1 wt %. The water content can be about 4±1 wt %. The water content can be about 4±0.75 wt %. The water content can be about 4±0.5 wt %. The water content can be about 4±0.25 wt %. The water content can be about 4±0.1 wt %. The water content can be about 4.5±1 wt %. The water content can be about 4.5±0.75 wt %. The water content can be about 4.5±0.5 wt %. The water content can be about 4.5±0.25 wt %. The water content can be about 4.5±0.1 wt %. The water content can be about 5±1 wt %. The water content can be about 5±0.75 wt %. The water content can be about 5±0.5 wt %. The water content can be about 5±0.25 wt %. The water content can be about 5±0.1 wt %. The water content can be about 5.5±1 wt %. The water content can be about 5.5±0.75 wt %. The water content can be about 5.5±0.5 wt %. The water content can be about 5.5±0.25 wt %. The water content can be about 5.5±0.1 wt %. The water content can be about 6±1 wt %. The water content can be about 6±0.5 wt %. The water content can be about 6.5±1 wt %. The water content can be about 6.5±0.5 wt %. The water content can be about 7±1 wt %. The water content can be about 7±0.5 wt %. The water content can be about 7.5±1 wt %. The water content can be about 7.5±0.5 wt %. The water content can be about 8±1 wt %. The water content can be about 8±0.5 wt %. The water content can be about 8.5±1 wt %. The water content can be about 8.5±0.5 wt %. The water content can be about 9±1 wt %. The water content can be about 9±0.5 wt %. The water content can be about 9.5±0.5 wt %. The water content can be about 9.5±0.25 wt %.

In some embodiments, crystalline Form H is substantially free of an organic solvent. In some embodiments, crystalline Form H is substantially free of any organic solvents. In some embodiments, Form H contains less than 5,000 ppm of a class III solvent, as defined by the U.S. Food and Drug Administration in Q3C Impurities: Residual Solvents. In some embodiments, Form H contains less than 5,000 ppm of any class III solvent, as defined by the U.S. Food and Drug Administration in Q3C Impurities: Residual Solvents. In some embodiments, Form H contains a concentration of a class II solvent which is below the accepted concentration as defined by the U.S. Food and Drug Administration in Q3C Impurities: Residual Solvents. In some embodiments, Form H contains a concentration of any class II solvent which is below the accepted concentration as defined by the U.S. Food and Drug Administration in Q3C Impurities: Residual Solvents. In some embodiments, Form H is substantially free of an ester. In some embodiments, Form H is substantially free of any esters. In some such embodiments, Form H has an ester content of less than about 5%, less than about 4%, less than about 3%, less than about 2%, less than about 1%, less than about 0.5%, less than about 0.1%, less than about 0.05%, less than about 0.02%, or less than about 0.01% by weight. In some embodiments, Form H is substantially free of ethyl acetate. In some such embodiments, Form H has an ethyl acetate content of less than about 5%, less than about 4%, less than about 3%, less than about 2%, less than about 1%, less than about 0.5%, less than about 0.1%, less than about 0.05%, less than about 0.02%, or less than about 0.01% by weight. In some embodiments, Form H is substantially free of a nonpolar solvent. In some embodiments, Form H is substantially free of any nonpolar solvents. In some such embodiments, Form H has a nonpolar solvent content of less than about 5%, less than about 4%, less than about 3%, less than about 2%, less than about 1%, less than about 0.5%, less than about 0.1%, less than about 0.05%, less than about 0.02%, or less than about 0.01% by weight. In some embodiments, Form H is substantially free of toluene. In some such embodiments, Form H has a toluene of less than about 5%, less than about 4%, less than about 3%, less than about 2%, less than about 1%, less than about 0.5%, less than about 0.1%, less than about 0.05%, less than about 0.02%, or less than about 0.01% by weight.

### Methods of Preparation

### Form H

In another aspect, provided herein are methods of preparing crystalline Form H of birinapant. In some embodiments, provided is a method of preparing crystalline Form H of birinapant comprising converting a first form of birinapant to a second form of birinapant via a conversion process, wherein the second form of birinapant is crystalline Form H of birinapant. In some embodiments, the first form of birinapant is Form D of birinapant (Form D). Form D is a stable, crystalline ethyl acetate solvate of birinapant. In some embodiments the method further comprises preparing the first form of birinapant via a formation process.

In some embodiments, the formation process comprises preparing a mixture of a protected birinapant derivative and a solvent, wherein the protected birinapant derivative comprises functional groups protected with acid-labile protecting groups; adding an acid to the mixture of the protected birinapant derivative and a solvent to remove the acid-labile protecting groups, thereby producing birinapant or a salt thereof; and neutralizing the acid.

In some embodiments, the formation process comprises preparing a mixture of Compound A

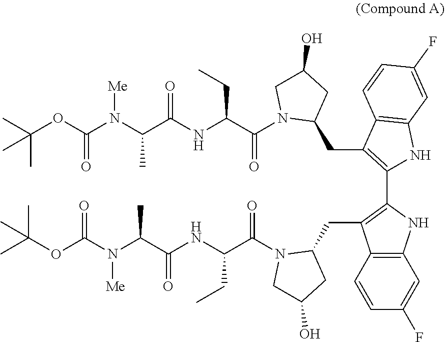 [SMILES: CC[C@H](NC(=O)[C@H](C)N(C)C(=O)OC(C)(C)C)C(=O)N1C[C@@H](O)C[C@H]1Cc1c(-c2[nH]c3cc(F)ccc3c2C[C@@H]2C[C@H](O)CN2C(=O)[C@H](CC)NC(=O)[C@H](C)N(C)C(=O)OC(C)(C)C)[nH]c2cc(F)ccc12]

and a solvent, wherein Compound A comprises amino groups protected with t-butyloxycarbonyl groups; adding an acid to the mixture of Compound A and a solvent to remove the t-butyloxycarbonyl groups, thereby producing birinapant or a salt thereof; and neutralizing the acid.

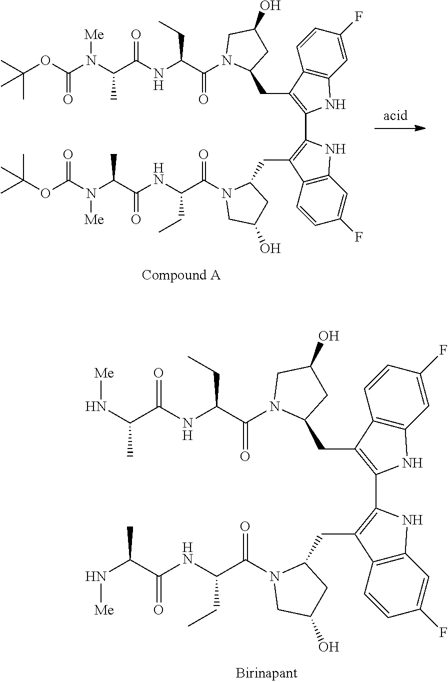 [SMILES: CC[C@H](NC(=O)[C@H](C)N(C)C(=O)OC(C)(C)C)C(=O)N1C[C@@H](O)C[C@H]1Cc1c(-c2[nH]c3cc(F)ccc3c2C[C@@H]2C[C@H](O)CN2C(=O)[C@H](CC)NC(=O)[C@H](C)N(C)C(=O)OC(C)(C)C)[nH]c2cc(F)ccc12 ; CC[C@H](NC(=O)[C@H](C)NC)C(=O)N1C[C@@H](O)C[C@H]1Cc1c(-c2[nH]c3cc(F)ccc3c2C[C@@H]2C[C@H](O)CN2C(=O)[C@H](CC)NC(=O)[C@H](C)NC)[nH]c2cc(F)ccc12]

In some embodiments, the formation process further comprises thermal cycling.

In some embodiments, the formation process comprises preparing a mixture of a protected derivative of birinapant and a solvent, and deprotecting the protected derivative of birinapant to produce the first form of birinapant. In some embodiments, the protected derivative of birinapant comprises t-butyloxycarbonyl protecting groups on the methylamino groups of birinapant, and deprotecting the protected derivative of birinapant comprises adding acid to the mixture of the protected form of birinapant and the solvent. In some such embodiments, the solvent comprises an ester and an alcohol. In some embodiments, the ester is ethyl acetate. In some embodiments, the alcohol is methanol. In some embodiments, the formation process further comprises subjecting the mixture to one or more temperature cycles. In some such embodiments, the one or more temperature cycles comprise a heating step. In some embodiments, the heating step comprises heating the mixture to a temperature of between about 40° C. and about 65° C., between about 40° C. and about 55° C., between about 50° C. and about 65° C., between about 40° C. and about 45° C., between about 45° C. and about 50° C., between about 50° C. and about 55° C., between about 55° C. and about 60° C., or between about 60° C. and about 65° C.. In one embodiment, the mixture is heated to about 55° C.+/−about 5° C. In some embodiments, the formation process comprises filtering the mixture. In some embodiments, the filtering generates filtered solids. In some embodiments, the formation process comprises drying the filtered solids to obtain Form D. Any methods known in the art may be used to dry the mixture. In some embodiments, the mixture is dried under vacuum at a temperature of between about 40° C. and about 65° C., between about 40° C. and about 55° C., between about 50° C. and about 65° C., between 40° C. and 45° C., between 45° C. and 50° C., between 50° C. and 55° C., between 55° C. and 60° C., or between 60° C. and 65° C.

In some embodiments, the formation process comprises preparing a mixture of Compound A and a solvent, and adding acid to the mixture.

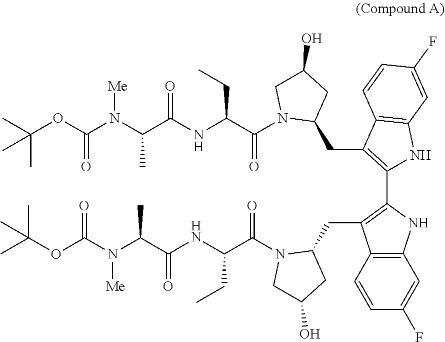 [SMILES: CC[C@H](NC(=O)[C@H](C)N(C)C(=O)OC(C)(C)C)C(=O)N1C[C@@H](O)C[C@H]1Cc1c(-c2[nH]c3cc(F)ccc3c2C[C@@H]2C[C@H](O)CN2C(=O)[C@H](CC)NC(=O)[C@H](C)N(C)C(=O)OC(C)(C)C)[nH]c2cc(F)ccc12]

In some such embodiments, the solvent comprises an alcohol. In some such embodiments, the alcohol is methanol. In some embodiments the formation process further comprises subjecting the mixture to one or more temperature cycles. In some such embodiments, the one or more temperature cycles comprise a first heating step, a first cooling step, a second heating step, and a second cooling step. In some such embodiments, the first heating step comprises heating the mixture to at least about 40° C., at least about 45° C., at least about 50° C., at least about 55° C., at least about 60° C., or at least about 65° C. In some embodiments the first heating step comprises heating the mixture to a temperature within about 40° C. to about 50° C., within about 45° C. to about 55° C., within about 50° C. to about 60° C., or within about 55° C. to about 65° C. In some embodiments, the first heating step comprises heating the mixture to between about 40° C. and about 65° C., between about 40° C. and about 55° C., between about 50° C. and about 65° C., between 40° C. and 45° C., between 45° C. and 50° C., between 50° C. and 55° C., between 55° C. and 60° C., or between 60° C. and 65° C. In one embodiment, the first heating step comprises heating the mixture to about 55° C.+/−about 5° C.

The formation process comprises adding an acid to the mixture. In some such embodiments, the formation process comprises adding an acid to the mixture during the first heating step. In some embodiments, the formation process comprises adding an acid to the mixture before the first heating step. In some embodiments, the formation process comprises adding an acid to the mixture after the first heating step. In some embodiments, the acid is hydrochloric acid. In some embodiments, the acid is concentrated hydrochloric acid. The acid can be added in

In some embodiments, the first cooling step comprises cooling the mixture to about 26° C., about 25° C., about 24° C., about 23° C., about 22° C., about 21° C., about 20° C., about 19° C., or about 18° C. In some embodiments, the first cooling step comprises cooling the mixture to a temperature of within about 5° C. (i.e., +/−about 5° C.) about 26° C., about 25° C., about 24° C., about 23° C., about 22° C., about 21° C., about 20° C., about 19° C., or about 18° C. In some embodiments, the first cooling step comprises cooling the mixture to a temperature of within about 10° C. (i.e., +/−about 10° C.) about 26° C., about 25° C., about 24° C., about 23° C., about 22° C., about 21° C., about 20° C., about 19° C., or about 18° C. In some embodiments, the first cooling step comprises cooling the mixture to a temperature between 25° C. and 20° C. or between 20° C. and 15° C. In some such embodiments, deionized (DI) water is added to the mixture during the first cooling step. In some embodiments, the DI water is added in a volume approximately 75% to 125% of the volume of the original of solvent used to prepare a mixture of Compound A and a solvent, such as about 100% of the volume of the original amount of solvent. In some embodiments, a base is added to the mixture following the first cooling step. In some embodiments, the base is added in an amount to adjust the pH to about 11+/−0.5. In some such embodiments, the base is sodium hydroxide, such as 2N sodium hydroxide. In some embodiments, the mixture is concentrated following the first cooling step. In some embodiments, the reaction mixture is extracted into an organic second solvent following the first cooling step and any concentration steps by adding the organic second solvent to the reaction mixture to form a biphasic mixture with an organic layer and an aqueous layer, with subsequent separation of the organic layer. In some such embodiments, the organic second solvent is an ester. In some such embodiments the ester is ethyl acetate. In some embodiments, the organic layer is concentrated under vacuum at a temperature of ≤45° C. In some embodiments, additional volumes of the organic second solvent can be added, followed by concentrated under vacuum.

In some embodiments, the second heating step comprises heating the organic layer to between about 30° C. to 40° C., such as to about 40° C., about 39° C., about 38° C., about 37° C., about 36° C., about 35° C., about 34° C., about 33° C., about 32° C., about 31° C., or about 30° C.

In some embodiments, the second cooling step comprises cooling the organic layer to between about 18° C. to about 26° C., or between about 20° C. to about 25° C., or between about 15° C. and 20° C. such as about 26° C., about 25° C., about 24° C., about 23° C., about 22° C., about 21° C., about 20° C., about 19° C., or about 18° C. In some embodiments, the formation process comprises filtering the organic layer. In some embodiments, the filtering generates filtered solids. In some such embodiments, the filtered solids are dried to obtain birinapant form D.

In some embodiments, the conversion process comprises preparing a mixture of birinapant form D and a solvent. In some such embodiments, the solvent comprises an alcohol. In some such embodiments, the alcohol is methanol. In some embodiments the conversion process further comprises subjecting the mixture to one or more temperature cycles. In some such embodiments, the one or more temperature cycles comprise a heating step and a cooling step.

In some such embodiments, the heating step in the conversion process comprises heating the mixture to at least about 40° C., at least about 41° C., at least about 42° C., at least about 43° C., at least about 44° C., at least about 45° C., at least about 46° C., at least about 47° C., at least about 48° C., at least about 49° C., at least about 50° C., at least about 51° C., at least about 52° C., at least about 53° C., at least about 54° C., at least about 55° C., at least about 56° C., at least about 57° C., at least about 58° C., at least about 59° C., at least about 60° C. at least about 61° C., at least about 62° C., at least about 63° C., at least about 64° C., or at least about 65° C. In some such embodiments, the heating step comprises heating the mixture to a temperature between about 40° C. and about 45° C., about 45° C. and about 50° C., about 50° C. and about 55° C., about 55° C. and about 60° C., or about 60° C. and about 65° C. Preferably, the heating step comprises heating the mixture to a temperature of about 55° C.+/−about 5° C.,

In some embodiments the cooling step in the conversion process comprises cooling the mixture to about 20° C., about 19° C., about 18° C., about 17° C., about 16° C., about 15° C., about 14° C., about 13° C., about 12° C., about 11° C., about 10° C., about 9° C., about 8° C., about 7° C., about 6° C., about 5° C., about 4° C., about 3° C., about 2° C., about 1° C., or about 0° C. In some embodiments the cooling step comprises cooling the mixture to a temperature within about 1° C. (i.e., +/−1° C.) of about 20° C., about 19° C., about 18° C., about 17° C., about 16° C., about 15° C., about 14° C., about 13° C., about 12° C., about 11° C., about 10° C., about 9° C., about 8° C., about 7° C., about 6° C., about 5° C., about 4° C., about 3° C., about 2° C., about 1° C., or about 0° C. In some embodiments the cooling step comprises cooling the mixture to a temperature within about 5° C. (i.e., +/−5° C.) of about 20° C., about 19° C., about 18° C., about 17° C., about 16° C., about 15° C., about 14° C., about 13° C., about 12° C., about 11° C., about 10° C., about 9° C., about 8° C., about 7° C., about 6° C., about 5° C., about 4° C., about 3° C., about 2° C., about 1° C., or about 0° C. In some embodiments the cooling step comprises cooling the mixture to a temperature within about 10° C. (i.e., +/−10° C.) of about 20° C., about 19° C., about 18° C., about 17° C., about 16° C., about 15° C., about 14° C., about 13° C., about 12° C., about 11° C., about 10° C., about 9° C., about 8° C., about 7° C., about 6° C., about 5° C., about 4° C., about 3° C., about 2° C., about 1° C., or about 0° C. In some embodiments, the cooling step comprises cooling the mixture to a temperature between about 0° C. and about 5° C., between about 5° C. and about 10° C., between about 10° C. and about 15° C., or between about 15° C. and about 20° C. In some such embodiments, the cooling step further comprises holding the mixture at an intermediate temperature between the final temperature of the heating step and the final temperature of the cooling step for a set period of time. In some embodiments, the intermediate temperature is between 15° C. and 20° C., between 20° C. and 25° C., between 25° C. and 30° C., between 30° C. and 35° C., or between 35° C. and 40° C. In some embodiments, the intermediate temperature is within about 1° C. (i.e., +/−1° C.) of about 40° C., about 39° C., about 38° C., about 37° C., about 36° C., about 35° C., about 34° C., about 33° C., about 32° C., about 31° C., about 30° C., about 29° C., about 28° C., about 27° C., about 26° C., about 25° C., about 24° C., about 23° C., about 22° C., about 21° C., about 20° C., about 19° C., or about 18° C. In some embodiments, the intermediate temperature is within about 5° C. (i.e., +/−5° C.) of about 40° C., about 39° C., about 38° C., about 37° C., about 36° C., about 35° C., about 34° C., about 33° C., about 32° C., about 31° C., about 30° C., about 29° C., about 28° C., about 27° C., about 26° C., about 25° C., about 24° C., about 23° C., about 22° C., about 21° C., about 20° C., about 19° C., or about 18° C. In some embodiments, the intermediate temperature is within about 10° C. (i.e., +/−10° C.) of about 40° C., about 39° C., about 38° C., about 37° C., about 36° C., about 35° C., about 34° C., about 33° C., about 32° C., about 31° C., about 30° C., about 29° C., about 28° C., about 27° C., about 26° C., about 25° C., about 24° C., about 23° C., about 22° C., about 21° C., about 20° C., about 19° C., or about 18° C. In some embodiments, the conversion process further comprises adding one or more seed crystals to the mixture to promote crystallization of Form H. In some such embodiments, the seed crystals comprise crystals of birinapant Form H. In some embodiments the seed crystals are added to the mixture during the cooling step. In some such embodiments, the seed crystals are added while the mixture is being held at the intermediate temperature during the cooling step. In some embodiments, the conversion process comprises filtering the mixture. In some embodiments, the filtering generates filtered solids. In some such embodiments, the conversion process further comprises drying the filtered solids to obtain Form H. Any methods known in the art may be used to dry the mixture. In some embodiments, the mixture is dried under vacuum under ambient temperature. In some embodiments, nitrogen gas is flowed over the mixture during drying.

### Pharmaceutical Compositions and Formulations

Pharmaceutical compositions of the crystalline form detailed herein are embraced by this invention. Thus, the invention includes pharmaceutical compositions comprising the crystalline form disclosed herein and a pharmaceutically acceptable carrier or excipient. In one embodiment, the pharmaceutical composition is a composition for controlled release of the crystalline form detailed herein.

In some embodiments, provided is a composition comprising Form H. In some embodiments, the composition is substantially free of amorphous or non-crystalline form of Birinapant or a salt thereof. In some embodiments of the composition comprising Form H, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of the total composition is Form H. In some embodiments of the composition comprising Form H, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of Birinapant exists in Form H.

Crystalline forms or compositions disclosed herein may be formulated for any available delivery route, including an oral, mucosal (e.g., nasal, sublingual, vaginal, buccal or rectal), parenteral (e.g., intramuscular, subcutaneous or intravenous), topical or transdermal delivery form, or a form suitable for inhalation. A crystalline form or composition disclosed herein may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

Crystalline forms disclosed herein can be used in the preparation of a formulation, such as a pharmaceutical formulation, by combining the crystalline form as an active ingredient with a pharmaceutically acceptable carrier, such as those mentioned above. Depending on the therapeutic form of the system (e.g., transdermal patch vs. oral tablet vs. solution for intravenous injection), the carrier may be in various forms. In addition, pharmaceutical formulations may contain preservatives, solubilizers, stabilizers, re-wetting agents, emulsifiers, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington: The Science and Practice of Pharmacy, Lippincott Williams & Wilkins, 21^{st} ed. (2005), which is incorporated herein by reference.

Crystalline forms disclosed herein may be administered to individuals (e.g., a human) in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulsifiers, sweeteners, dyes, adjusters, and salts for the adjustment of osmotic pressure, buffers, coating agents or antioxidants.

Birinapant can be administered by intravenous injection, including, e.g., by infusion over about 1 to about 120 minutes, e.g., about 30 minutes. Birinapant can be prepared in unit dose form, i.e., a form suitable for single administration to a subject, and can be supplied in an intravenous unit dose form, such as a vial or pre-filled syringe with a pre-measured amount of birinapant. The effective dose administered in each injection is an amount that is effective and tolerated; it can be, e.g., 0.01 to 30 mg/m², e.g., 0.2 to 10 mg/m², or, e.g., 0.5 to 5 mg/m². An effective dose is one that over the course of therapy results in treatment of the proliferative disorder.

In various embodiments, a dose, e.g., a unit dose, such as a unit dose for weekly administration, can be a solution formed from dissolving the crystalline form in a solvent. The solution can comprise about 0.1, 0.25, 0.5, 0.75, 1, 2.5, or 5 milligrams per mL. Additional dosing ranges and regiments, and concentration ranges, are disclosed in U.S. Pat. No. 8,283,372 and US Patent Publication No. 2014/0243276.

The dosage form for administration can be administered to an individual in need thereof once weekly. That is, the total amount of birinapant that is to be administered each week can be administered all together at one time once per week. Alternatively, if it is desirable that the total amount of birinapant is to be administered in two or more portions weekly, the dosage form containing the appropriate amount of crystalline form can be administered two times or more per week, such as twice per week, three times per week, or four times per week. Alternatively, the dosage form can be administered once every ten days, once every two weeks, or on a four-week treatment cycle with a dosage administered once weekly for three consecutive weeks, followed by a week without dosing (i.e., a treatment cycle of three weeks “on” and one week “off”).

Compositions comprising a crystalline form disclosed herein are also described. In some embodiments, the composition is for use as a human or veterinary medicament. In some embodiments, the composition is for use in a method described herein. In some embodiments, the composition is for use in the treatment of a disease or disorder described herein.

### Methods of Use

Crystalline forms and compositions disclosed herein may be used in methods of administration and treatment as provided herein. The crystalline forms and compositions may also be used in in vitro methods, such as in vitro methods of administering a compound or composition to cells for screening purposes and/or for conducting quality control assays.

In some embodiments, provided is a method of treating a cell proliferative disorder in an individual in need thereof comprising administering to the individual a therapeutically effective amount of the crystalline form disclosed herein. In some embodiments, the individual is a human. The individual, such as human, may be in need of treatment, such as a human who has or is suspected of having a cell proliferative disorder.

In some embodiments, provided is a crystalline form for use in the treatment of a cell proliferative disorder. Also provided is use of a crystalline form in the manufacture of a medicament for the treatment of a cell proliferative disorder. Treatment of a cell proliferative disorder using birinapant is described in U.S. Pat. Nos. 8,283,372, 8,603,816, 8,986,993, 10,034,912, 10,314,881, and 10,596,220, which are hereby incorporated by reference herein in their entirety.

In any of the described methods, in one aspect the individual is a human, such as a human in need of the method. The individual may be a human who has been diagnosed with or is suspected of having a cell proliferative disorder. The individual may be a human who does not have detectable disease but who has one or more risk factors for developing a cell proliferative disorder.

In any of the described methods, in one aspect the cell proliferative disorder is cancer. In some embodiments, the cancer is a hematologic cancer or a solid tumor. In some embodiments, the cancer is a hematologic cancer. In some embodiments, the hematologic cancer is leukemia, lymphoma, myeloma, any metastases thereof, or any combination thereof. In some embodiments, the hematologic cancer is acute myeloid leukemia (AML), chronic myeloid leukemia (CML), acute lymphocytic leukemia (ALL), small lymphocytic lymphoma (SLL), chronic lymphocytic leukemia, hairy cell leukemia, Hodgkin lymphoma, non-Hodgkin lymphoma, multiple myeloma, any metastases thereof, or any combination thereof. In some embodiments, the hematologic cancer is acute myeloid leukemia (AML). In some embodiments, the cancer is a solid tumor. In some embodiments, the cancer is bladder cancer, colorectal cancer, sarcoma, gastric cancer, lung cancer, pancreatic cancer, melanoma, ovarian cancer, cervical cancer, head and neck cancer, kidney cancer, liver cancer, or breast cancer. In some embodiments, the cancer is sarcoma. In some embodiments, the sarcoma is fibrosarcoma, chondrosarcoma, or osteosarcoma. In some embodiments, the sarcoma is fibrosarcoma. In some embodiments, the cancer is lung cancer. In some embodiments, the lung cancer is non-small cell lung cancer (NSCLC). In some embodiments, the cancer therapy comprises carboplatin. In some embodiments, the cancer therapy comprises paclitaxel. In some embodiments, the cancer is head and neck cancer. In some embodiments, the head and neck cancer is head and neck sarcoma. In some embodiments, the head and neck cancer is esophageal cancer. In some embodiments, the cancer is breast cancer. In some embodiments, the breast cancer is breast carcinoma. In some embodiments, the breast cancer is triple negative breast cancer (TNBC).

### Kits

In another aspect, provided is a kit comprising crystalline form disclosed herein, a solution formed by dissolving the crystalline form herein, or a pharmaceutical composition comprising a crystalline form as described herein. The kits may employ any of the crystalline forms disclosed herein. The kits may be used for any one or more of the uses described herein, and, accordingly, may contain instructions for use in the treatment of a cell proliferative disorder.

Kits generally comprise suitable packaging. The kits may comprise one or more containers comprising any compound described herein. Each component (if there is more than one component) can be packaged in separate containers or some components can be combined in one container where cross-reactivity and shelf life permit. One or more components of a kit may be sterile and/or may be contained within sterile packaging.

The kits may be in unit dosage forms, bulk packages (e.g., multi-dose packages) or sub-unit doses. For example, kits may be provided that contain sufficient dosages of a compound as disclosed herein (e.g., a therapeutically effective amount) and/or a second pharmaceutically active compound useful for a disease detailed herein (e.g., cancer) to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the crystalline forms and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to an individual.

### EXAMPLES

The following examples are provided to further aid in understanding the embodiments disclosed in the application, and presuppose an understanding of conventional methods well known to those persons having ordinary skill in the art to which the examples pertain. The particular materials and conditions described hereunder are intended to exemplify particular aspects of embodiments disclosed herein and should not be construed to limit the reasonable scope thereof.

The following abbreviations may be used herein:

| | |
|---|---|
| XRPD | X-Ray Powder Diffraction |
| VT-XRPD | Variable Temperature X-Ray Powder Diffraction |
| DSC | Differential Scanning Calorimetry |
| TGA | Thermogravimetric Analysis |
| DVS | Dynamic Vapor Sorption |
| VS | Vapor Sorption |
| LCMS | Liquid Chromatography Mass Spectrometry |
| ¹H NMR | Proton Nuclear Magnetic Resonance |
| FID | Free Induction Decay |
| KF | Karl Fischer |
| equiv. or eq. | Equivalents |
| DI | Deionized |
| RH | Relative humidity |
| RT | Room temperature |
| EtOAc | Ethyl acetate |
| MeOH | Methanol |
| DMSO | Dimethylsulfoxide |
| API | Active Pharmaceutical Ingredient |
| HPLC | High performance liquid chromatography |
| UPLC | Ultra performance liquid chromatography |

The crystalline forms were characterized by various analytical techniques, including XRPD, DSC, TGA, DVS, 1H NMR, and HPLC using the procedures described below.

### XRPD

XRPD patterns were collected with a PANalytical Empyrean diffractometer using an incident beam of Cu radiation produced using a long, fine-focus source. An elliptically graded multilayer mirror was used to focus Cu Kα X-rays through the specimen and onto the detector. Prior to analysis, a silicon specimen (NIST SRM 640e) was analyzed to verify the observed position of the Si 111 peak is consistent with the NIST-certified position. A specimen of the sample was sandwiched between 3-μm-thick films and analyzed in transmission geometry. A beam-stop, short antiscatter extension, and antiscatter knife edge were used to minimize the background generated by air. Soller slits for the incident and diffracted beams were used to minimize broadening and asymmetry from axial divergence. Diffraction patterns were collected using a scanning position-sensitive detector (X′Celerator) located 240 mm from the specimen and Data Collector software v. 5.5.

Parameters for XRPD test

| Parameter | Value |
|---|---|
| Scan axis: | Gonio |
| Scan range (°): | 1.0000-40.0041 |
| Start position (°): | 1.0084 |
| End position (°): | 39.9957 |
| Step size (°): | 0.0167 |
| No. of points: | 2334 |
| Scan mode: | Continuous |
| Phi (°): | 257.3 |
| Test time (s) | About 12 min 1 s |

### DSC

DSC was performed using a Mettler-Toledo DSC3+ differential scanning calorimeter. A tau lag adjustment was performed with indium, tin, and zinc. The temperature and enthalpy were adjusted with octane, phenyl salicylate, indium, tin, and zinc. The adjustment was then verified with octane, phenyl salicylate, indium, tin, and zinc. The sample was placed into a hermetically sealed aluminum DSC pan, and the weight was recorded. The pan was then inserted into the DSC cell. A weighed aluminum pan configured as the sample pan was placed on the reference side of the cell. The pan lid was pierced prior to sample analysis. Samples were analyzed from −30° C. to 350° C. at 10°/min.

### TGA

TGA analysis was performed using a Mettler-Toledo TGA/DSC3 analyzer. Temperature and enthalpy adjustments were performed using indium, tin, and zinc, and then verified with indium. The balance was verified with calcium oxalate. The sample was placed in an open aluminum pan. The pan was hermetically sealed, the lid pierced, and then inserted into the TG furnace. A weighed aluminum pan configured as the sample pan was placed on the reference platform. The furnace was heated under nitrogen. Each sample was heated from ambient temperature to 350° C. at 10° C./min. The thermograms were plotted by reference temperature (x-axis) and results were reported according to sample temperatures.

### DVS

Automated vapor sorption (VS) data were collected on a Surface Measurement System DVS Intrinsic instrument. Samples were not dried prior to analysis. Sorption and desorption data were collected over a range from 5% to 95% RH at 10% RH increments under a nitrogen purge. The equilibrium criterion used for analysis was less than 0.0100% weight change in 5 minutes with a maximum equilibration time of 3 hours. Data were not corrected for the initial moisture content of the samples.

### ¹H NMR

The ¹H NMR spectrum was acquired with Bruker AVANCE NEO spectrometer at a ¹H Larmor frequency of 600.134 MHz. The spectrum was acquired at 25° C. with a 1H pulse width of 10.6 μs, a 5.0 second acquisition time, a 25 second delay between scans, a spectral width of 10000 Hz, 10000 data points, and 4 co-added scans. The free induction decay (FID) was processed using Bruker TopSpin 4.0.8 software with 524288 points and an exponential line broadening factor of 0.2 Hz to improve the signal-to-noise ratio. The spectra were referenced to the tetramethylsilane peak at 0.0 ppm.

### Example 1. Preparation and Characterization of Birinapant Form H

Birinapant form H was prepared by first preparing birinapant Form D, and then converting birinapant Form D to birinapant Form H. Detailed descriptions of each process are given below.

Birinapant form D was prepared using the following process. First, in a 5-L, jacketed reactor, Compound A (structure shown below) (168.3 g, 0.167 moles, 1.0 equiv) and MeOH (1.18 L, 7 vol) were charged.

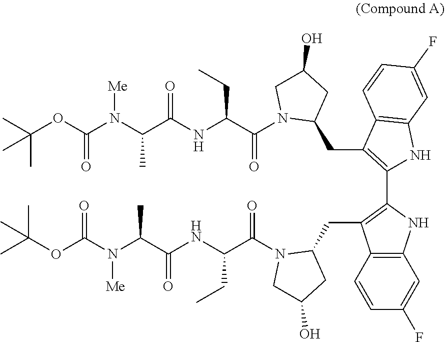 [SMILES: CC[C@H](NC(=O)[C@H](C)N(C)C(=O)OC(C)(C)C)C(=O)N1C[C@@H](O)C[C@H]1Cc1c(-c2[nH]c3cc(F)ccc3c2C[C@@H]2C[C@H](O)CN2C(=O)[C@H](CC)NC(=O)[C@H](C)N(C)C(=O)OC(C)(C)C)[nH]c2cc(F)ccc12]

The resulting white suspension was heated to 55±5° C. A clear solution formed at 35.4° C. Concentrated HCl (82 mL, 6.0 equiv) was charged dropwise (over a period of 43 min) to avoid foaming of the batch. The resulting clear greenish-yellow solution was analyzed by HPLC, showing the presence of 81.98% birinapant, 17.18% of intermediate, and 0.83% of Compound A. The batch was then heated at 55±5° C. and heating was stopped. HPLC analysis showed 100% birinapant. The batch was then cooled to ambient temperature over 1.5 h. DI water (1.18 L, 7 vol) was charged into the batch at 20-25° C. over a period of 25 min. The batch turned into a dark-green solution and was left at ambient temperature overnight under N₂ (no stirring). The batch was concentrated to 9.0 vol (≈1500 mL) at 40-45° C. by vacuum distillation. After cooling to 20-25° C., EtOAc (1.180 L, 7 vol) was added slowly over a period of 5 min. 50% NaOH (65 mL) was added slowly at 20±5° C. to adjust the pH of the batch from 0.88 to ≈10.18. At pH of 6.3 the mixture turned into a yellowish-brown biphasic solution. The two-phase system was stirred for 5 min at ambient temperature and then was allowed to stand for phase separation. The aqueous layer and the EtOAc layers were isolated and kept separate. The aqueous phase was re-extracted with EtOAc (1.180 L). The aqueous layer was isolated, and the EtOAc extracts were combined and washed with 5% NaHCO₃ (420 mL, 2.5 vol). The batch was stirred for 5 min and then allowed to stand at ambient temperature for phase separation. The phase separation occurred in 6 min. The EtOAc layer and the aqueous layer were separated. A thin rag layer was observed and was kept with the aqueous layer. The EtOAc layer was stored at ambient temperature overnight. The reactor was cleaned with water, MeOH, and EtOAc, then was dried overnight under N₂ sweep. The EtOAc layer was polish filtered into the clean and dry reactor through a 0.3-μm, in-line filter. The residue left in the container was rinsed with 340 mL EtOAc and this was added into the batch through the in-line filter. KF analysis of the EtOAc layer showed presence of 4.32% water. The EtOAc layer was concentrated to ≈9 vol (≈1500 mL) by vacuum distillation at 40-45° C. To the reactor was charged with another 1.68 L of EtOAc and concentrated to 5.8 vol (≈980 mL). At this stage, the KF analysis showed presence of 0.23% water (water spec: ≤0.1%). Vacuum distillation was performed two more times by adding fresh EtOAc (1.68 L) and concentrating the mixture to ≈1000 mL. KF analysis showed presence of 0.21% and 0.20% water respectively after the additional two vacuum distillations (water spec: ≤0.1%). Since the two additional vacuum distillations did not reduce water level any further, it was decided to move to the next step.

The batch was heated at 35° C. for 30 min, then cooled to 20° C., and stirred at 20° C. overnight. The suspension was filtered and the filter cake was washed with EtOAc (2×1.5 vol, 2×252 mL). The filtrate and washings were combined and kept aside. The filter cake was dried under high vacuum at ambient temperature over 72 h to afford 135.6 g, yield: 93% of birinapant Form D. 1H NMR was consistent with the desired structure and HPLC analysis showed purity of 99.6% (AUC). LC-MS showed the desired [M+1] peak XRPD analysis showed the desired D form of birinapant.

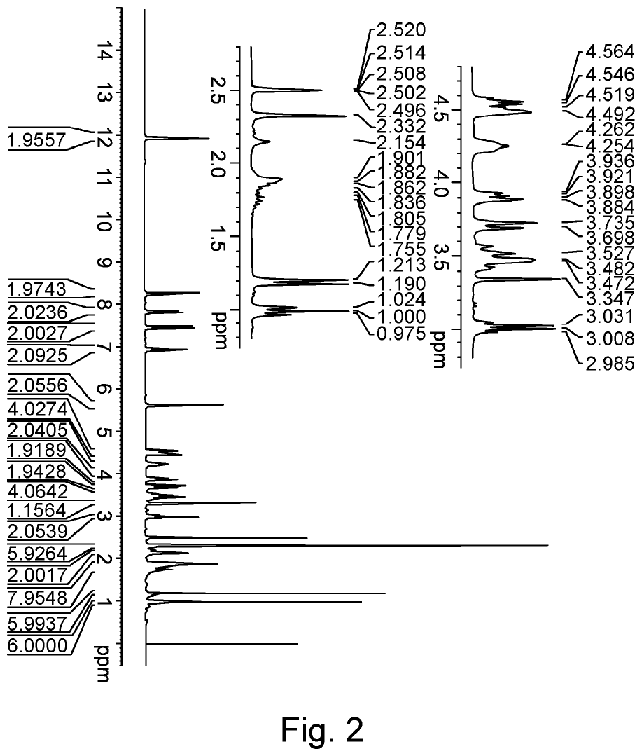

Birinapant Form D was then converted into birinapant form H via the following process. First, a slurry of birinapant Form D (130 g corresponding to 119.6 g corrected for 7.7% EtOAc and 0.6% water) in MeOH (250 mL, 1.9 vol) was prepared in a clean Erlenmeyer flask. The slurry was then charged into a 1-L, jacketed reactor. The Erlenmeyer flask was rinsed with 270 mL MeOH (2.1 vol). The rinse was added to the batch and was stirred at ambient temperature to obtain a uniform suspension. The batch was then heated to 55±5° C., and at 42.8° C. a clear solution formed. The batch was next cooled to 35° C. over a period of 15 min and was seeded with 1.3 g, 0.01 wt equiv (Birinapant Form H). The crystals of Birinapant Form H used for seeding were prepared according to the same process described herein, but without a seeding step. The seed persisted. The batch was cooled to 5±5° C. over a period of 1 h and was stirred at 6° C. overnight. The clear yellow solution turned into a white suspension. The batch was filtered, the reactor was rinsed with cold methanol (1 vol, 130 mL), and the rinse was used to wash the filter cake. The filtrate and washing were combined and kept aside. The filter cake was dried under high vacuum at ambient temperature with a nitrogen bleed to obtain 98.67 g, recovery: 82% corrected for 0.6% water and 7.7% EtOAc in the birinapant Form D starting material, and 0.84% water in birinapant Form H. ¹H NMR (FIG. 2) was consistent with the desired structure, LCMS showed peak corresponding to [M+1], and UPLC analysis showed purity of 99.46% (AUC). XRPD analysis showed the desired H form of birinapant (FIG. 1A).

### Example 2. Reaction Optimization for the Synthesis of Birinapant Form H

Several different procedures for the conversion of birinapant form D to birinapant form H were also tested. The tested procedures and the resulting yields and observations are presented in Table 2, below.

TABLE 2

| Process | | | Results and |
|---|---|---|---|
| No. | Reaction Conditions | IPC Stage | Comments |
| 1 | 4.3 g, birinapant | Chase residual solvent with | 54% yield, Form H of |
| | MeOH 5 vol | MeOH 5 vol × 2; Dissolve in | birinapant, no residual |
| | T 50→15° C., 5 h | methanol at 50° C., slowly cooled | solvent such as MeOH |
| | | to 20° C. over 5 h, seeded with | and EtOAc |
| | | form H at 20° C. | |
| 2 | 10 g, birinapant | Chase residual solvent with | 81% yield, Form H of |
| | MeOH 5 vol | MeOH 5 vol × 2; Dissolve in | birinapant, no residual |
| | T 50→15° C., 5 h | methanol at 50° C., slowly cooled | solvent such as MeOH |
| | | to 20° C. over 5 h, seeded with | and EtOAc |
| | | form H at 30° C. | |
| 3 | 5 g, birinapant | Dissolve in methanol at 50° C., | 80% yield, Form H of |
| | MeOH 5 vol | and slowly cooled to 20° C. over | birinapant, no residual |
| | T 50→15° C., 5 h | 5 h, seeded with form H at | solvent such as MeOH |
| | | 28° C. | and EtOAc |
| 4 | 2 g, birinapant | Dissolve in methanol at 50 ± | Recovery: 64.6% |
| | MeOH 5 vol | 5° C., slowly cooled to 5 ± 5° C., | (corrected for 0.94% |
| | T 50 ± 5° C. → 5 ± 5° C. | and maintained at 5 ± 5° C. | water) Form H of |
| | 5 ± 5° C. overnight | overnight, seeded with form H at | birinapant |
| | | 35° C. | |
| 5 | 2 g, birinapant | Dissolve in methanol at 50 ± | Recovery: 64.6% |
| | MeOH 4 vol | 5° C., slowly cooled to 5 ± 5° C., | (corrected for 0.94% |
| | T 50 ± 5° C. → 5 ± 5° C. | and maintained at 5 ± 5° C. | water) Form H of |
| | 5 ± 5° C. overnight | overnight, seeded with form H at | birinapant |
| | | 30 ± 5° C., filter cake was | |
| | | washed with 1 vol of cold | |
| | | MeOH. | |

### Example 3. Stability of Birinapant Form H

A stability study of birinapant form H was performed by leaving the material open to air at room temperature and exposed to light for one month. The surface of the solid discolored slightly during this interval. HPLC analysis indicated that the purity of birinapant remained at 99.9% after a period of one month. Further, Karl Fischer titration analysis indicated that the water content of the material remained consistently between 0.3 to 0.4% over the course of the one-month study.

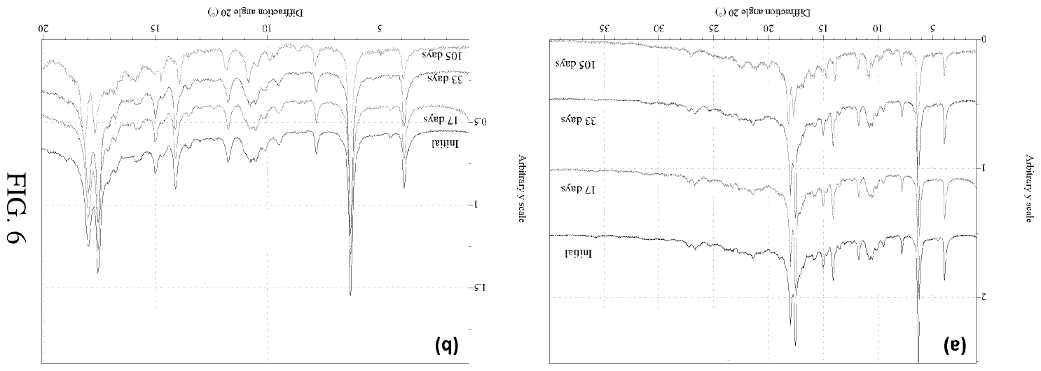

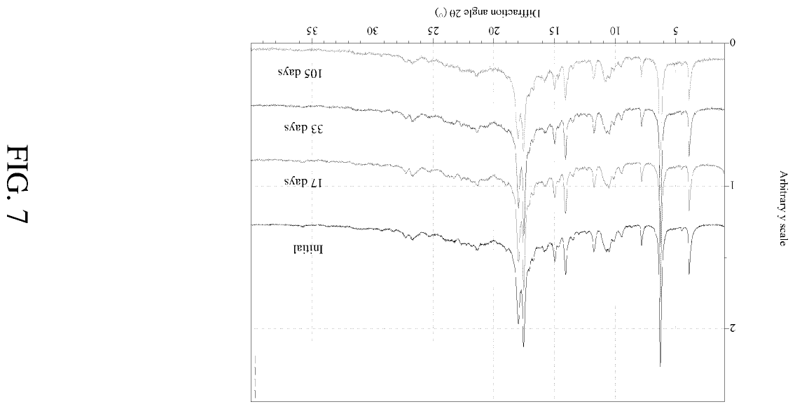

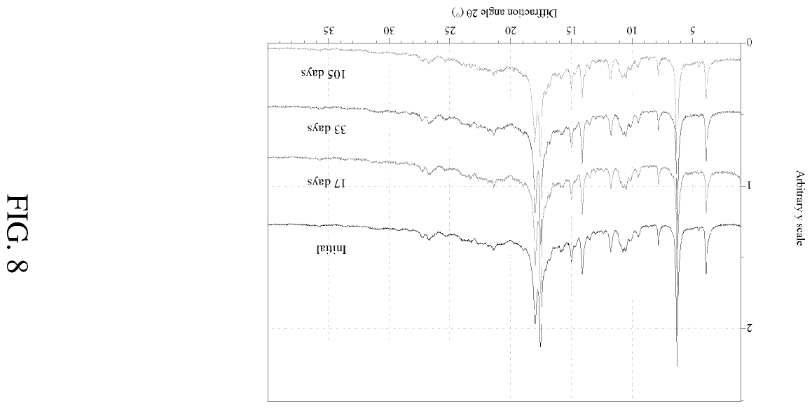

Additional stability studies under elevated temperature and relative humidity (RH) conditions were also performed. Samples of birinapant form H were subjected to various temperature and humidity conditions for up to 105 days, and samples were taken at 17, 33, and 105 days. For each sample the identity of the crystalline form of the sample was assessed using XRPD and the birefringence of the sample was assessed using polarized light microscopy. The results of these experiments are summarized in Table 3, below. Additionally, XRPD patterns for several time points are provided for the 60° C. study, the 30° C./62% RH study, and the 40° C./75% RH study in FIGS. 6, 7, and 8, respectively. The time points shown are at the start of the study (top pattern), after 17 days (pattern second from top), after 33 days (pattern third from top), and after 105 days (bottom pattern).

TABLE 3

| | | | | Resulting |
|---|---|---|---|---|
| Conditions^{a} | Time | | Observation^{b} | form |
| 60° C. | 17 | d | fines, B | H |
| | 33 | d | white to off-white in color | H |
| | 105 | d | off-white | H |
| 80° C. | 17 | d | fines, B sample faint pale yellow | H |
| | 33 | d | pale yellow in color | H |
| 245° C. | 1 | hr | light tan solids | C |
| RT, 53% RH | 18 | d | fines, B sample faint pale yellow | H |
| 30° C., | 17 | d | fines, B | H |
| 62% RH | 33 | d | — | H |
| | 105 | d | white fines, B | H |
| 40° C., | 17 | d | fines, B | H |
| 75% RH | 33 | d | white solids | H |
| | 105 | d | white fines, B | H |
| RT, 98% RH | 18 | d | fines, B sample faint pale yellow | H |

^{a}Reported time, temperature, and humidity are approximate. RT = Room temperature.
^{b}B = birefringence and NB = no birefringence upon observation by polarized light microscopy.

### Example 4. DSC and VT-XRPD Study of Birinapant Form H

A DSC study was performed on a sample of birinapant Form H. The results of this study are shown in FIG. 3A. A sample of birinapant Form H was heated from about 20° C. to about 400° C. At about 175° C., a slight endothermic peak was observed, corresponding to the melting of birinapant Form H. At about 180° C., an exothermic peak was observed, corresponding to recrystallization of birinapant into Form A. A second exothermic peak at about 230° C. corresponded to conversion of Form A to Form C. The large endothermic peak at about 320° C. corresponded to the melting of Form C, followed by a broad endothermic feature between about 340° C. and about 400° C., which corresponded to the thermal decomposition of birinapant. The VT-XRPD pattern shown in FIG. 3B shows the X-ray diffraction peaks for each of the aforementioned forms, and also for Form Z, a birinapant polymorph formed at high temperatures.

### Example 5. TGA Study of Birinapant Form H

A TGA study was performed on a sample of birinapant Form H. The results of this study are shown in FIG. 4. Briefly, a sample of biripant Form H was heated from about 25° C. to about 400° C. No weight loss was observed between about 25° C. and 300° C. Between about 340° C. and 400° C., weight loss was observed, corresponding to the thermal decomposition of birinapant.

### Example 6. DVS Study of Birinapant Form H

A DVS study was performed on a sample of birinapant Form H. The results of this study are shown in FIG. 5. This study was performed at a temperature of 25° C., and the relative humidity was cycled between about 5% and about 95%. The data collected during this study is given in Table 4, below.

TABLE 4

| | Cycle 1 Sorp | | Cycle 1 Desorp | | |
|---|---|---|---|---|---|
| Target | Sample | Sorp Mass | Sample | Desorp Mass | |
| RH (%) | RH (%) | Change (%) | RH (%) | Change (%) | Hysteresis |
| 5.0 | 5.1 | 0.000 | 5.5 | 0.020 | |
| 15.0 | 15.6 | 0.365 | 16.1 | 0.557 | 0.192 |
| 25.0 | 25.1 | 0.732 | 25.6 | 0.929 | 0.197 |
| 35.0 | 34.7 | 1.001 | 35.3 | 1.238 | 0.237 |
| 45.0 | 45.0 | 1.224 | 45.6 | 1.532 | 0.308 |
| 55.0 | 54.7 | 1.436 | 55.4 | 1.815 | 0.379 |
| 65.0 | 64.7 | 1.652 | 65.5 | 2.078 | 0.425 |
| 75.0 | 74.7 | 1.908 | 75.3 | 2.336 | 0.428 |
| 85.0 | 84.9 | 2.263 | 86.0 | 2.610 | 0.347 |
| 95.0 | 95.7 | 2.904 | 95.7 | 2.904 | |

### Example 7. Stability of Birinapant Form H

A stability study of birinapant form H was performed by leaving the material open to air at room temperature and exposed to light for one month. The surface of the solid discolored slightly during this interval. HPLC analysis indicated that the purity of birinapant remained at 99.9% after a period of one month. Further, Karl Fischer titration analysis indicated that the water content of the material remained consistently between 0.3 to 0.4% over the course of the one-month study.

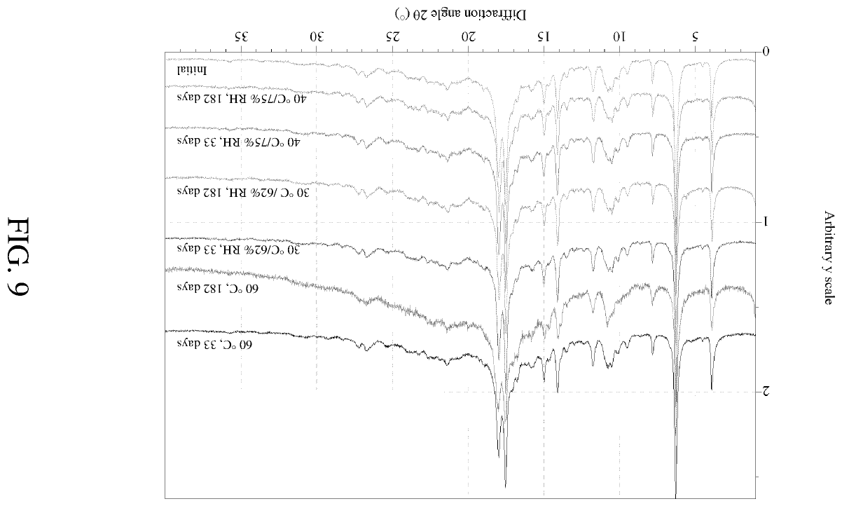

Additional stability studies under elevated temperature and relative humidity (RH) conditions were also performed. Samples of birinapant form H were subjected to various temperature and humidity conditions for up to 182 days, and samples were taken at 17, 33, 105, and 183 days. For each sample the identity of the crystalline form of the sample was assessed using XRPD and the birefringence of the sample was assessed using polarized light microscopy. The results of these experiments are summarized in Table 5, below. The “RESULT” column indicates the form that was observed. Additionally, XRPD patterns for several time points are provided for the 60° C. study, the 30° C./62% RH study, and the 40° C./75% RH study in FIG. 9. The spectra shown in FIG. 9 correspond to the 60° C. study after 33 days (top trace), the 60° C. study after 182 days (trace second from top), the 30° C./62% RH study after 33 days (trace third from top), the 30° C./62% RH study after 182 days (trace fourth from top), the 40° C./75% RH study after 33 days (trace fifth from top), and the 40° C./75% RH study after 182 days (trace sixth from top). A representative XRPD trace for birinapant form H prior to stressing is also shown for comparison (bottom trace).

TABLE 5

| Condition^{a} | Time | | Observation^{b} | Result |
|---|---|---|---|---|
| 60° C. | 17 | d | fines, B | H |
| | 33 | d | white to off-white in color | H |
| | 105 | d | off-white | H |
| | 182 | d | off-white to pale yellow^{c} | H |
| 80° C. | 17 | d | fines, B sample faint pale yellow | H |
| | 33 | d | pale yellow in color | H |
| 245° C. | 1 | hr | light tan solids | C |
| RT, 53% RH | 18 | d | fines, B sample faint pale yellow | H |
| 30° C., 62% RH | 17 | d | fines, B | H |
| | 33 | d | — | H |
| | 105 | d | white fines, B | H |
| | 182 | d | white solids^{d} | H |
| 40° C., 75% RH | 17 | d | fines, B | H |
| | 33 | d | white solids | H |
| | 105 | d | white fines, B | H |
| | 182 | d | white solids^{e} | H |
| RT, 98% RH | 18 | d | fines, B sample faint pale yellow | H |

^{a}Reported time, temperature, and humidity are approximate. RT = Room temperature.
^{b}B = birefringence and NB = no birefringence upon observation by polarized light microscopy.
^{c}temperature excursions 1) 105° C. 3 hours on day 113 and 2) power outage 1 hour on day 125
^{d}temperature excursion due to power outage, duration 1 hour on day 125
^{e}temperature excursion due to power outage, duration 1 hour on day 125

All references throughout, such as publications, patents, patent applications and published patent applications, are incorporated herein by reference in their entireties.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

What is claimed is:

1. A crystalline form of birinapant, wherein the crystalline form is characterized as having an XRPD pattern comprising a peak at an angle 2-theta of about 6.47.

2. The crystalline form of claim 1, wherein the crystalline form is characterized as having an XRPD pattern comprising peaks at angles 2-theta of about 6.47, about 17.67, and about 18.20.

3. The crystalline form of claim 1 or claim 2, wherein the crystalline form is characterized as having an XRPD pattern comprising:
a. peaks at angles 2-theta of about 6.47, about 10.6, about 10.82, about 15.94, about 16.19, about 17.67, about 18.20, and about 18.67 degrees; or
b. peaks at angles 2-theta of about 6.47, about 10.67, about 10.82, about 15.94, about 16.19, about 17.67, about 18.20, and about 18.67 degrees; or
c. peaks at angles 2-theta of about 4.03, about 6.09, about 6.47, about 10.67, about 10.82, about 15.04, about 15.94, about 16.19, about 17.67, about 18.20, and about 18.67 degrees; or
d. peaks at angles 2-theta of about 3.17, about 4.03, about 4.35, about 4.49, about 4.70, about 4.96, about 5.09, about 5.40, about 5.81, about 6.09, about 6., about 7.15, about 8.07, about 8.77, about 9.01, about 9.15, about 10.04, about 10.67, about 10.82, about 12.66, about 13.24, about 13.98, about 15.04, about 15.94, about 16.19, about 17.67, about 18.20, about 18.67, about 21.04, about 22.88, about 23.63, about 24.70, about 25.57, about 26.16, about 28.39, about 29.59, about 30.22, about 31.58, and about 40.84 degrees.

4. The crystalline form of any one of claims 1-3, wherein the crystalline form is characterized as having an XRPD pattern substantially as shown in FIG. 1A or FIG. 1B

5. The crystalline form of any one of claims 1-4, wherein the crystalline form is characterized as having a DSC graph substantially as shown in FIG. 3A.

6. A crystalline form of birinapant, wherein the crystalline form is characterized as having a melting point between about 171° C. and about 177° C.

7. The crystalline form of any one of claims 1-5, wherein the crystalline form is characterized as having a melting point between about 171° C. and about 177° C.

8. The crystalline form of any one of claims 1-7, wherein the crystalline form is characterized as having a TGA graph substantially as shown in FIG. 4.

9. The crystalline form of any one of claims 1-8, wherein the crystalline form is characterized as showing no weight loss after heating from about 25° C. to about 300° C. as determined by TGA.

10. The crystalline form of any one of claims 1-9, wherein the crystalline form has a water content of less than 2% by weight.

11. The crystalline form of any one of claims 1-10, wherein the crystalline form is anhydrous.

12. The crystalline form of any one of claims 1-11, wherein the crystalline form is characterized as having a DVS graph substantially as shown in FIG. 5.

13. A method of preparing the crystalline form of any one of claims 1-12, comprising converting a first form of birinapant to a second form of birinapant via a conversion process; wherein the second form of birinapant is the crystalline form of any one of claims 1-12.

14. The method of claim 13, further comprising preparing a first form of birinapant via a formation process.

15. The method of claim 14, wherein the formation process comprises preparing a formation mixture of Compound A: 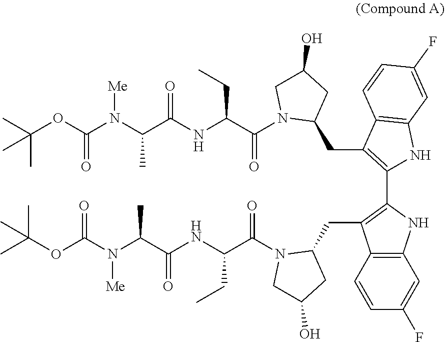 [SMILES: CC[C@H](NC(=O)[C@H](C)N(C)C(=O)OC(C)(C)C)C(=O)N1C[C@@H](O)C[C@H]1Cc1c(-c2[nH]c3cc(F)ccc3c2C[C@@H]2C[C@H](O)CN2C(=O)[C@H](CC)NC(=O)[C@H](C)N(C)C(=O)OC(C)(C)C)[nH]c2cc(F)ccc12]
and a formation solvent.

16. The method of claim 15, wherein the formation solvent comprises an alcohol.

17. The method of claim 16, wherein the alcohol is methanol.

18. The method of any one of claims 15-17, further comprising adding an acid to the formation mixture, thereby deprotecting Compound A to produce birinapant.

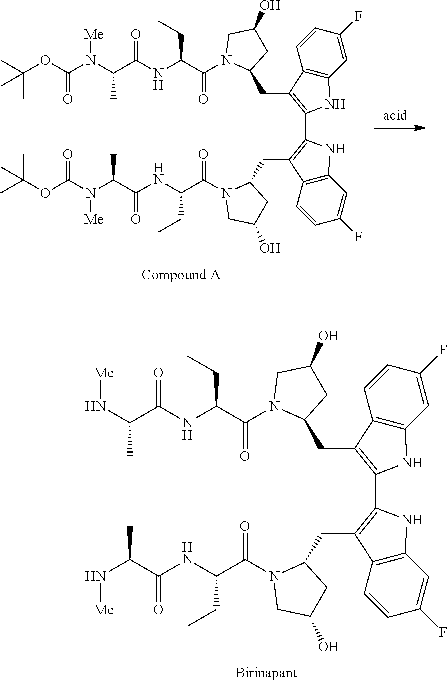 [SMILES: CC[C@H](NC(=O)[C@H](C)N(C)C(=O)OC(C)(C)C)C(=O)N1C[C@@H](O)C[C@H]1Cc1c(-c2[nH]c3cc(F)ccc3c2C[C@@H]2C[C@H](O)CN2C(=O)[C@H](CC)NC(=O)[C@H](C)N(C)C(=O)OC(C)(C)C)[nH]c2cc(F)ccc12 ; CC[C@H](NC(=O)[C@H](C)NC)C(=O)N1C[C@@H](O)C[C@H]1Cc1c(-c2[nH]c3cc(F)ccc3c2C[C@@H]2C[C@H](O)CN2C(=O)[C@H](CC)NC(=O)[C@H](C)NC)[nH]c2cc(F)ccc12]

19. The method of claim 18, wherein the acid is hydrochloric acid.

20. The method of any one of claims 15-19, wherein the formation process further comprises subjecting the formation mixture to a temperature cycle.

21. The method of claim 20, wherein the formation temperature cycle comprises heating the formation mixture to between about 50° C. to about 60° C., then cooling the formation mixture to between about 20° C. to about 25° C.

22. The method of claim 21, further comprising adding water to the formation mixture.

23. The method of any one of claims 18-22, further comprising adding an organic second solvent to the formation mixture.

24. The method of claim 23, wherein the organic second solvent is an ester.

25. The method of claim 24, wherein the ester is ethyl acetate.

26. The method of any one of claims 18-25, further comprising adding base to the formation mixture.

27. The method of claim 26, further comprising allowing the formation mixture to stand to form a biphasic mixture with an organic layer and an aqueous layer.

28. The method of claim 27, further comprising separating the organic layer and the aqueous layer.

29. The method of claim 28, further comprising concentrating the organic layer.

30. The method of any one of claims 27-29, wherein the formation process further comprises subjecting the organic layer to a second heating step and then a second cooling step, thereby producing a precipitate comprising the first form of birinapant.

31. The method of claim 30, wherein the formation process further comprises drying the precipitate at 50° C. under vacuum.

32. The method of any one of claims 13-31, wherein the conversion process comprises preparing a conversion mixture of the first form of birinapant and a conversion solvent.

33. The method of claim 32, wherein the conversion solvent comprises an alcohol.

34. The method of claim 33, wherein the conversion solvent comprises methanol.

35. The method of any one of claims 32-34, wherein the conversion process further comprises subjecting the conversion mixture to a temperature cycle.

36. The method of claim 35, wherein the conversion temperature cycle comprises the steps of
a. heating the conversion mixture to about 50° C.+/−about 5° C.;
b. cooling the conversion mixture to about 35° C.+/−about 5° C.; and
c. cooling the conversion mixture to about 5° C.+/−about 5° C.

37. A crystalline form of birinapant prepared according to the method of any one of claims 13-36.

38. A method of manufacturing a pharmaceutical composition comprising birinapant, comprising combining the crystalline form of any one of claim 1-12 or 37 and a pharmaceutically acceptable excipient.

39. A pharmaceutical composition comprising the crystalline form of any one of claim 1-12 or 37, and a pharmaceutically acceptable carrier or excipient.

40. A pharmaceutical composition prepared according to the method of claim 38.

41. A kit comprising the crystalline form of any one of claim 1-12 or 37, or the pharmaceutical composition of claim 39 or 40.

42. The kit of claim 41, further comprising instructions for the treatment of a cell proliferative disorder.

43. The kit of any one of claim 41 or 42, wherein the cell proliferative disorder is cancer.

44. A method of treating a cell proliferative disorder in an individual in need thereof comprising administering to the individual a therapeutically effective amount of the crystalline form of any one of claim 1-12 or 37, or the pharmaceutical composition of claim 39 or 40.

45. The method of claim 44, wherein the cell proliferative disorder is cancer.

46. Use of the crystalline form of any one of claim 1-12 or 37 in the manufacture of a medicament for the treatment of a cell proliferative disorder.

47. The use of claim 46, wherein the cell proliferative disorder is cancer.

48. The crystalline form of any one of claim 1-12 or 37, or the pharmaceutical composition of claim 39 or 40, for use in the treatment of a cell proliferative disorder.

49. The crystalline form of any one of claim 1-12 or 37, or the pharmaceutical composition of claim 39 or 40, for use in the treatment of cancer.
